SEQUENCE LISTING (38 sequences)

SEQ ID NO: 1 - DNA, 135 bp, Artificial Sequence
  Synthetic sequence
  misc_feature: Homo sapiens CD8alpha hinge - DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 2 - protein, 45 aa, Artificial Sequence
  Synthetic sequence
  MISC_FEATURE: Homo sapiens CD8a hinge - protein
TTTPAPRPPTPAPTIASQPLSLRPEACRPAAGGAVHTRGLDFACD

SEQ ID NO: 3 - DNA, 63 bp, Artificial Sequence
  Synthetic sequence
  misc_feature: Homo sapiens CD8 TM - DNA
atctacatctgggcgcccttggccgggacttgtggggtccttctcctgtcactggttatcacc

SEQ ID NO: 4 - protein, 21 aa, Artificial Sequence
  Synthetic sequence
  MISC_FEATURE: Homo sapiens CD8 TM - protein
IYIWAPLAGTCGVLLLSLVIT

SEQ ID NO: 5 - DNA, 111 bp, Artificial Sequence
  Synthetic sequence
  misc_feature: Homo sapiens OX40 - DNA
cggagggaccagaggctgccccccgatgcccacaagccccctgggggaggcagtttccggacccccatccaagaggagcaggccgacgcccactccaccctggccaagatc

SEQ ID NO: 6 - protein, 37 aa, Artificial Sequence
  Synthetic sequence
  MISC_FEATURE: Homo sapiens OX40 - protein
RRDQRLPPDAHKPPGGGSFRTPIQEEQADAHSTLAKI

SEQ ID NO: 7 - DNA, 336 bp, Artificial Sequence
  Synthetic sequence
  misc_feature: Homo sapiens CD3zeta - DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - protein, 112 aa, Artificial Sequence
  Synthetic sequence
  MISC_FEATURE: Homo sapiens CD3zeta - protein
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, Artificial Sequence
  Synthetic sequence
  misc_feature: Homo sapiens T2A - DNA
ggctctggcgagggaaggggttccctgcttacttgcggcgacgtcgaagagaatcccggtccg

SEQ ID NO: 10 - protein, 21 aa, Artificial Sequence
  Synthetic sequence
  MISC_FEATURE: Homo sapiens T2A - Protein
GSGEGRGSLLTCGDVEENPGP

SEQ ID NO: 11 - DNA, 342 bp, Artificial Sequence
  Synthetic sequence
  misc_feature: Homo sapiens native IL15 - DNA
aactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtcc

SEQ ID NO: 12 - protein, 114 aa, Artificial Sequence
  Synthetic sequence
  MISC_FEATURE: Homo sapiens native IL15 - protein
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 13 - DNA, 207 bp, Artificial Sequence
  Synthetic sequence
  misc_feature: Homo sapiens CD8hinge/TM - DNA
actaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 14 - protein, 66 aa, Artificial Sequence
  Synthetic sequence
  MISC_FEATURE: Homo sapiens CD8hinge/TM - protein
TTTPAPRPPTPAPTIASQPLSLRPEACRPAAGGAVHTRGLDFACDIYIWAPLAGTCGVLLLSLVIT

SEQ ID NO: 15 - DNA, 30 bp, Artificial Sequence
  Synthetic sequence
  misc_feature: Homo sapiens Linker 1 (used after FLAG tag) DNA
ggcggtggtggctctggtggtggcggcagc

SEQ ID NO: 16 - protein, 10 aa, Artificial Sequence
  Synthetic sequence
  MISC_FEATURE: Homo sapiens Linker 1 (used after FLAG tag) Protein
GGGGSGGGGS

SEQ ID NO: 17 - DNA, 30 bp, Artificial Sequence
  Synthetic sequence
  misc_feature: Homo sapiens Linker 2 - after neg. control binding domain - DNA
ggccaggccggctccggaggaggaggatcc

SEQ ID NO: 18 - protein, 10 aa, Artificial Sequence
  Synthetic sequence
  MISC_FEATURE: Homo sapiens Linker 2 - after neg. control binding domain - protein
GQAGSGGGGS

SEQ ID NO: 19 - DNA, 12 bp, Artificial Sequence
  Synthetic sequence
  misc_feature: Homo sapiens Linker post scFv - DNA
ggccaggccggc

SEQ ID NO: 20 - protein, 4 aa, Artificial Sequence
  Synthetic sequence
  MISC_FEATURE: Homo sapiens Linker post scFv - protein
GQAG

SEQ ID NO: 21 - DNA, 33 bp, Artificial Sequence
  Synthetic sequence
  misc_feature: Homo sapiens Misc Linker - DNA
ggcggcggcggtagcggtggtggcggctccgga

SEQ ID NO: 22 - protein, 11 aa, Artificial Sequence
  Synthetic sequence
  MISC_FEATURE: Homo sapiens Misc Linker - Protein
GGGGSGGGGSG

SEQ ID NO: 23 - DNA, 18 bp, Artificial Sequence
  Synthetic sequence
  misc_feature: Homo sapiens Linker after 09 Homing moiety - DNA
ggccaggccggctccgga

SEQ ID NO: 24 - protein, 6 aa, Artificial Sequence
  Synthetic sequence
  MISC_FEATURE: Homo sapiens Linker after 09 Homing moiety - Protein
GQAGSG

SEQ ID NO: 25 - DNA, 405 bp, Artificial Sequence
  Synthetic sequence
  misc_feature: Homo sapiens NKG2D Extracellular Fragment - DNA
ttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 26 - protein, 135 aa, Artificial Sequence
  Synthetic sequence
  MISC_FEATURE: Homo sapiens NKG2D Extracellular Fragment - Protein
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 27 - DNA, 645 bp, Artificial Sequence
  Synthetic sequence
  misc_feature: Homo sapiens Full Human NKG2D - DNA
gggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtaacaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 28 - DNA, 405 bp, Artificial Sequence
  Synthetic sequence
  misc_feature: Codon optimized human NKG2D fragment - DNA
ctgttcaatcaggaagtccagatccccctgacagagtcttactgcggcccatgtcccaagaactggatctgctacaagaacaattgttatcagttctttgacgagagcaagaactggtatgagtcccaggcctcttgcatgagccagaatgcctctctgctgaaggtgtacagcaaggaggaccaggatctgctgaagctggtgaagtcctatcactggatgggcctggtgcacatccctacaaacggctcttggcagtgggaggacggctccatcctgtctccaaatctgctgaccatcatcgagatgcagaagggcgattgcgccctgtacgccagctccttcaagggctatatcgagaactgctccacacccaatacctacatctgtatgcagaggaccgtg

SEQ ID NO: 29 - DNA, 126 bp, Artificial Sequence
  Synthetic sequence
  misc_feature: Homo sapiens 4-1BB - DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 30 - protein, 42 aa, Artificial Sequence
  Synthetic sequence
  MISC_FEATURE: Homo sapiens 4-1BB - Protein
KRGRKKLLYIFKQPFMRPVQTTQEEDGCSCRFPEEEEGGCEL

SEQ ID NO: 31 - protein, 29 aa, Artificial Sequence
  Synthetic sequence
  MISC_FEATURE: Homo sapiens Amino Acid Sequence CD28 Transmembrane domain
FWVLVVVGGVLACYSLLVTVAFIIFWVRS

SEQ ID NO: 32 - protein, 39 aa, Artificial Sequence
  Synthetic sequence
  MISC_FEATURE: Homo sapiens Amino Acid Sequence CD28 IC domain
KRSRLLHSDYMNMTPRRPGPTRKHYQPYAPPRDFAAYRS

SEQ ID NO: 33 - DNA, 1122 bp, Artificial Sequence
  Synthetic sequence
  misc_feature: NKG2D-Ox40-CD3z - DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - protein, 374 aa, Artificial Sequence
  Synthetic sequence
  MISC_FEATURE: NKG2D-Ox40-CD3z - Protein
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 612 bp, Artificial Sequence
  Synthetic sequence
  misc_feature: mbIL15 - DNA
atggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 36 - protein, 204 aa, Artificial Sequence
  Synthetic sequence
  MISC_FEATURE: mbIL15 - Protein
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 37 - DNA, 614 bp, Artificial Sequence
  Synthetic sequence
  misc_feature: mbIL15 Option 2 - DNA
atggccttaccagtgaccgccttgctcctgccgctggccttgctgctccacgccgccaggccgaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggtatcaccctttactgctaa

SEQ ID NO: 38 - protein, 204 aa, Artificial Sequence
  Synthetic sequence
  MISC_FEATURE: mbIL15 Option 2 - Protein
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVSPFTA

(19) United States
(12) Patent Application Publication
(10) Pub. No.: US 2025/0073266 A1
(43) Pub. Date: Mar. 6, 2025

(54) DOSING REGIMENS FOR CANCER IMMUNOTHERAPY

(71) Applicant: NKARTA, INC., South San Francisco, CA (US)

(72) Inventors: Kanya Lakshmi Rajangam, San Francisco, CA (US); Minoti Murughesh Hiremath, Millbrae, CA (US); James Barnaby Trager, Albany, CA (US); David Richard Shook, Pacifica, CA (US); Aaron Norvin Endsley, Redmond, WA (US)

(21) Appl. No.: 18/559,514

(22) PCT Filed: May 11, 2022
(86) PCT No.: PCT/US2022/028839

§ 371 Date: Nov. 7, 2023

Related U.S. Application Data
(60) Provisional application No. 63/201,792, filed on May 13, 2021.
(60) Provisional application No. 63/269,316, filed on Mar. 14, 2022.

(51) Int. Cl. A61K 35/17; A61K 31/675; A61K 31/7068; A61K 31/7076; A61K 39/00; A61P 35/00; C07K 14/725

(52) U.S. Cl. CPC A61K 35/17; A61K 31/675; A61K 31/7068; A61K 31/7076; A61K 40/15; A61K 40/32; A61K 40/35; A61K 40/4224; A61P 35/00; C07K 14/7051; A61K 2239/38; A61K 2239/48; C12N 2510/00

(57) ABSTRACT

Several embodiments of the methods and compositions disclosed herein relate to immune cells that are engineered to express cytotoxic chimeric receptors and various dosing regimens for administering such cells. In several embodiments, the immune cells express a chimeric receptor that targets ligands of NKG2D on tumor cells. In several embodiments, the cancer is a blood cancer, for example, acute myeloid leukemia (e.g., relapsed/refractory acute myeloid leukemia) or myelodysplastic syndrome. In several embodiments, the tumor is a solid tumor, for example, intrahepatic cholangiocarcinoma or other liver tumor, for example, secondary metastases from colorectal cancer.

### CROSS REFERENCE TO RELATED APPLICATIONS

This application claims the benefit of priority to U.S. Provisional Patent Application No. 63/201,792, filed May 13, 2021 and U.S. Provisional Patent Application No. 63/269,316, filed Mar. 14, 2022, the entire contents of each of which is incorporated by reference herein.

### FIELD

Several embodiments disclosed herein relate to methods and compositions comprising genetically engineered cells for cancer immunotherapy. In several embodiments, the present disclosure relates to cells engineered to express cytotoxic receptor complexes and administration of such cells in accordance with certain dosing regimens to achieve successful cancer immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN ASCII TEXT FILE

This application incorporates by reference the Sequence Listing contained in the following ASCII text file being submitted concurrently herewith: File name: NKT.078NP_ST25.txt; created on May 14, 2024 and is 27,856 bytes in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric receptors in immune cells to achieve the targeted recognition and destruction of aberrant cells of interest.

In several embodiments, there is provided herein a population of genetically engineered natural killer (NK) cells for cancer immunotherapy, comprising a plurality of NK cells that have been expanded in culture, wherein the plurality of NK cells are engineered to express a cytotoxic receptor complexes comprising an NKG2D ligand binding domain, a transmembrane domain, and a cytotoxic signaling complex.

In several embodiments, there is provided a dosing regimen for cancer immunotherapy, comprising at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, wherein the third dose is administered to the subject between 5-10 days after the second dose, wherein each of the first, second and third doses comprise at least 1.0×10⁹ NK cells, wherein at least a portion of the engineered NK cells is engineered to express a chimeric receptor that binds ligands of the natural killer cell group 2D (NKG2D). In several embodiments, additional doses may be added to a dosing cycle, such as a fourth, fifth or greater dose.

In several embodiments, there are provided methods for the treatment of cancer, comprising administering to the subject at least a first, a second, and a third dose of genetically engineered NK cells, wherein the second dose is administered to the subject between 6-8 days after the first dose, wherein the third dose is administered to the subject between 6-8 days after the second dose, wherein each of the first, second and third doses comprise at least 1.0×10⁹ NK cells, and wherein the engineered NK cells are allogeneic with respect to the subject and are engineered to express a chimeric receptor that binds ligands of the natural killer cell group 2D (NKG2D).

In several embodiments, there are provided uses of a population of engineered NK cells expressing a chimeric receptor that targets ligands of the NKG2D receptor for treating cancer, by administration of at least a first, a second, and a third dose of the genetically engineered NK cells, wherein the second dose is administered to the subject between 6-8 days after the first dose, wherein the third dose is administered to the subject between 6-8 days after the second dose, and wherein each of the first, second and third doses comprise at least 1.0×10⁹ engineered NK cells.

In several embodiments, the first, second, and third dose each comprise about 1.5×10⁹ NK cells. In several embodiments, the first, second, and third dose each comprise at least 1.5×10⁹ NK cells. In several embodiments, the first, second, and third dose each comprise greater cell numbers, such as 2×10⁹ NK cells, 3×10⁹ NK cells, 4×10⁹ NK cells, 5×10⁹ NK cells, or more, such as 1 or 1.5×10⁹ NK cells.

In several embodiments, the dosing cycle is between about 14 days and about 35 days, such as about 21 days or about 28 days. In several embodiments, the first, second, and third doses of engineered NK cells are administered to the subject within about 21 days of the first time point. In several embodiments, the first, second, and third doses of engineered NK cells are administered to the subject within about 14 days after the first time point.

According to some embodiments, the first dosing cycle is initiated after the subject has undergone a lymphodepletion process in order to reduce native immune cell numbers. Depending on the embodiment, the first dosing cycle is optionally followed by one or more additional dosing cycle, such as two, three, four or more additional cycles. The additional cycles may be administered depending on the state of the cancer in a subject, e.g., in the event of progression or development of an additional cancer. In several embodiments, an additional cycle is not needed when a subject exhibits a complete response (e.g., is cancer free).

In several embodiments, the dosing regimen and related methods and uses comprise administering to a subject having cancer a lymphodepletion regimen comprising at least two doses of fludarabine. In several embodiments, the lymphodepletion process comprises at least two doses of cyclophosphamide and at least two doses of fludarabine. In several embodiments, the lymphodepletion process comprises three doses of cyclophosphamide and three doses of fludarabine, wherein the first of the doses of cyclophosphamide and fludarabine are administered 5 days prior to the initiation of the dosing cycle, wherein the second of the doses of cyclophosphamide and fludarabine are administered 4 days prior to the initiation of the dosing cycle, and wherein the third of the doses of cyclophosphamide and fludarabine are administered 3 days prior to the initiation of the dosing cycle. In several embodiments, about two days are allowed to lapse between the third dose of cyclophosphamide and fludarabine and initiation of the dosing cycle. In several embodiments, the cyclophosphamide is administered in an amount between about 50 and about 1000 mg/m² and the fludarabine is administered in an amount between about 5 and about 100 mg/m². In several embodiments, the cyclophosphamide is administered in an amount between about 100 and about 600 mg/m² and the fludarabine is administered in an amount between about 10 and about 60 mg/m². In several embodiments, the cyclophosphamide is administered in an amount between about 200 and about 400 mg/m² and the fludarabine is administered in an amount between about 20 and about 40 mg/m². In several embodiments, the cyclophosphamide is administered in an amount of about 300 mg/m² (e.g., about 250-350 mg/m²) and the fludarabine is administered in an amount of about 30 mg/m² (e.g., about 25-25 mg/m²). In several embodiments, the lymphodepletion process comprises at least two doses of cytosine arabinoside (Ara-C) and at least two doses of fludarabine. In several embodiments, the lymphodepletion process comprises 5 daily doses of Ara-C and 5 daily doses of fludarabine, wherein the first of the doses of Ara-C and fludarabine are administered 7 days prior to the initiation of the dosing cycle. In several embodiments, about two days are allowed to lapse between the final doses of Ara-C and fludarabine and initiation of the dosing cycle. In several embodiments, the Ara-C is administered in an amount between about 0.1 and about 20 g/m²/day and the fludarabine is administered in an amount between about 5 and about 100 mg/m²/day. In several embodiments, the Ara-C is administered in an amount between about 0.5 and about 10 g/m²/day and the fludarabine is administered in an amount between about 10 and about 60 mg/m²/day. In several embodiments, the Ara-C is administered in an amount between about 1 and about 5 g/m²/day and the fludarabine is administered in an amount between about 20 and about 40 mg/m²/day. In several embodiments, the Ara-C is administered in an amount of about 2 g/m²/day (e.g., about 1.5-2.5 g/m²/day) and the fludarabine is administered in an amount of about 30 mg/m²/day (e.g., about 25-35 mg/m²/day).

In several embodiments, the first and second doses of engineered NK cells are administered to the subject prior to the subject's native immune cell population recovering from the lymphodepletion process. Depending on the embodiment, other lymphodepletion agents may be used in addition to, or in place of cyclophosphamide, Ara-C and/or fludarabine, such as for example, daunorubicin (daunomycin) or idarubicin, mycophenolate mofetil, and/or bendamustine.

In several embodiments, the dosing regimen and related methods and uses are configured to treat a subject wherein the cancer is a blood cancer. In several embodiments, the cancer is Relapsed/Refractory (R/R) Acute Myeloid Leukemia (AML) or Higher Risk Myelodysplastic Syndrome (MDS). In several embodiments, the subject has R/R AML and has received at least 1 but not more than 2 lines of previous standard anti-leukemia therapy prior to the dosing regimen. In several embodiments, the subject has fms-like tyrosine kinase 3 (FLT3)-mutated and/or isocitrate dehydrogenase (IDH)1/2-mutated disease and has received at least 1, but not more than 3, lines of prior therapy prior to the dosing regimen. In several embodiments, the subject is classified as intermediate, high, or very high risk MDS and has relapsed and/or refractory MDS. In several embodiments, the subject has received at least 1 but not more than 2 lines of previous standard anti-MDS therapy prior to the dosing regimen. In several embodiments, the subject has less than about 5-10%, less than about 5-8%, or less than about 5% blasts in a blood sample prior to the dosing regimen. In several embodiments, the subject has a white blood cell count of less than or equal to 30×10⁹ WBC/L, less than or equal to 28×10⁹ WBC/L, or less than or equal to about 25×10⁹ WBC/L prior to the dosing regimen. In several embodiments, the subject does not exhibit evidence of leukemic meningitis or known active central nervous system disease and/or does not have peripheral leukocytosis with greater than or equal to 20,000 blasts/μL.

In several embodiments, the dosing regimen and related methods and uses are configured to treat a subject wherein the cancer is a solid tumor. In several embodiments, the cancer is a liver tumor, including intrahepatic cholangiocarcinoma, and liver tumors that are secondary metastases from colorectal cancer.

In several embodiments, the engineered NK cells express a chimeric receptor encoded by a polynucleotide having at least 85%, at least 90%, at least 95%, or at least 98% sequence identity to SEQ ID NO: 33. In several embodiments, the engineered NK cells express a chimeric receptor having at least 85%, at least 90%, at least 95%, or at least 98% sequence identity to SEQ ID NO: 34. In several embodiments, the engineered NK cells are also engineered to express membrane-bound interleukin 15 (mbIL15). In several embodiments, the mbIL15 has at least 85%, at least 90%, at least 95%, or at least 98% sequence identity to SEQ ID NO: 36 and/or 38. In several embodiments, the engineered NK cells are allogeneic with respect to the subject. In several embodiments, the dosing regimen, related methods and/or uses results in one or more of a reduction in blast cell counts, an increase in platelet counts, and an increase in neutrophil counts.

In several embodiments, there is provided a use of a population of engineered NK cells expressing a chimeric receptor that targets ligands of the NKG2D receptor for treating cancer in a subject, by intravenous administration of at least three sequential doses of genetically engineered NK cells, wherein the first dose of genetically engineered NK cells is administered to the subject at a first time point and comprises at least 1.0×10⁹ engineered NK cells, wherein the second dose is administered to the subject between 6-8 days after the first dose and comprises at least 1.0×10⁹ engineered NK cells, wherein the third dose is administered to the subject between 6-8 days after the second dose and comprises at least 1.0×10⁹ engineered NK cells, wherein the first time point is about two days after the conclusion of a lymphodepletion process comprising either (i) three doses of cyclophosphamide and three doses of fludarabine, wherein the cyclophosphamide is administered in an amount of about 300 mg/m² and the fludarabine is administered in an amount of about 30 mg/m², or (ii) 5 daily doses of Ara-C and 5 daily doses of fludarabine, wherein the Ara-C is administered in an amount of about 2 g/m²/day and the fludarabine is administered in an amount of about 30 mg/m²/day, and wherein the engineered NK cells express a chimeric receptor having at least 95% sequence identity to SEQ ID NO: 34. In several embodiments, each of the first, second and third doses comprise at least 1.5×10⁹ NK cells.

In several embodiments, there is provided a dosing regimen for cancer immunotherapy, comprising at least a first dosing cycle, wherein the first dosing cycle comprises a first dose of genetically engineered natural killer (NK) cells, a second dose of genetically engineered NK cells, and a third dose of genetically engineered NK cells. In several embodiments, the first dose is administered to a subject in need of cancer immunotherapy at a first time point, the second dose is administered to the subject between 5-10 days after the first time point, and the third dose is administered to the subject between 5-10 days after the second dose. In several embodiments, each of the first, second and third doses comprise at least 1.5×10⁹ NK cells (or at least 3×10⁷/kg for subject under 50 kg) and at least a portion of the engineered NK cells is engineered to express a chimeric receptor comprising a domain that binds ligands of the natural killer cell group 2D (NKG2D), a transmembrane domain, and a cytotoxic signaling complex. In several embodiments, the cytotoxic signaling complex comprises an OX40 subdomain and a CD3zeta subdomain. In several embodiments, the genetically engineered NK cells also express membrane-bound interleukin 15 (mbIL15). In several embodiments, the first dosing cycle is initiated after the subject has undergone a lymphodepletion process in order to reduce native immune cell numbers and is optionally followed by one or more additional dosing cycle.

Also provided for herein is a dosing regimen for cancer immunotherapy, comprising at least a first dosing cycle, wherein the first dosing cycle comprises a first dose of genetically engineered NK cells, a second dose of genetically engineered NK cells, and a third dose of genetically engineered NK cells with the first dose is administered to a subject at a first time point, the second dose being administered to the subject between 5-10 days after the first time point, the third dose being administered to the subject between 5-10 days after the second dose, and with each of the first, second and third doses comprising at least 1.5×10⁹ NK cells, at least a portion of which express a chimeric receptor comprising a domain that binds ligands of the natural killer cell group 2D (NKG2D). In several embodiments, the first dosing cycle is initiated after the subject has undergone a lymphodepletion process and is optionally followed by one or more additional dosing cycle.

In several embodiments, the dosing cycle is between about 14 days and about 35 days. In several embodiments, the dosing cycle is about 21 days or about 28 days (including 21, 22, 23, 24, 25, 26, 27, or 28 days). In several embodiments, the lymphodepletion process comprises at least two doses of cytosine arabinoside (Ara-C) and at least two doses of fludarabine. In several embodiments, the lymphodepletion process comprises 3, 4, or 5 daily doses of Ara-C and 3, 4, or 5 daily doses of fludarabine, wherein the first of the doses of Ara-C and fludarabine are administered 5 to 7 days prior to the initiation of the dosing cycle. In several embodiments, one, two, or three days are allowed to lapse between the final doses of Ara-C and fludarabine and initiation of the dosing cycle. In several embodiments, the Ara-C is administered in an amount between about 0.2 and 20 g/m²/day (e.g., 0.5 and 10 g/m²/day) and the fludarabine is administered in an amount between about 5 and 75 mg/m²/day (e.g., 10 and 60 mg/m²/day). In several embodiments, the Ara-C is administered in an amount between about 1 and 5 g/m²/day and the fludarabine is administered in an amount between about 20 and 40 mg/m²/day. In several embodiments, the Ara-C is administered in an amount of about 2 g/m²/day and the fludarabine is administered in an amount of about 30 mg/m²/day.

In several embodiments, the lymphodepletion process comprises at least two doses of cyclophosphamide and at least two doses of fludarabine. In several embodiments, the lymphodepletion process comprises three doses of cyclophosphamide and three doses of fludarabine, wherein the first of the doses of cyclophosphamide and fludarabine are administered 5 days prior to the initiation of the dosing cycle, wherein the second of the doses of cyclophosphamide and fludarabine are administered 4 days prior to the initiation of the dosing cycle, and wherein the third of the doses of cyclophosphamide and fludarabine are administered 3 days prior to the initiation of the dosing cycle. In several embodiments, one, two, or three days are allowed to lapse between the third dose of cyclophosphamide and fludarabine and initiation of the dosing cycle. In several embodiments, the cyclophosphamide is administered in an amount between about 10 and 1000 mg/m² (e.g., about 100 and 600 mg/m²) and the fludarabine is administered in an amount between about 5 and 100 mg/m² (about 10 and 60 mg/m²). In several embodiments, the cyclophosphamide is administered in an amount between about 200 and 400 mg/m² and the fludarabine is administered in an amount between about 20 and 40 mg/m². In several embodiments, the cyclophosphamide is administered in an amount of about 500 mg/m² and the fludarabine is administered in an amount of about 30 mg/m².

In several embodiments, the first and second doses of engineered NK cells are administered to the subject prior to the subject's native immune cell population recovering from the lymphodepletion process. In several embodiments, the first, second, and third doses of engineered NK cells are administered to the subject within about 21 days of the first time point. In several embodiments, the first, second, and third doses of engineered NK cells are administered to the subject within about 14 days after the first time point.

Additionally provided is a dosing regimen comprising at least a first dosing cycle, wherein the first dosing cycle is made up of a first, a second, and a third dose of genetically engineered NK cells, wherein at least a portion of the engineered NK cells is engineered to express a chimeric receptor comprising a domain that binds ligands of the natural killer cell group 2D (NKG2D), a transmembrane domain, and a cytotoxic signaling complex, wherein the first dose of the cycle is administered to a subject in need of cancer immunotherapy at a first time point, the second dose being administered to the subject between 5-10 days after the first time point, and the third dose being administered to the subject between 11-16 days after the first time point, resulting in, in combination, first, second, and third doses result administration of about 4 billion of the engineered NK cells. In several embodiments, the first, second, and third dose each comprise about 1.5×10⁹ NK cells.

In several embodiments of the dosing regimens provided for herein, the engineered NK cells express a chimeric receptor encoded by a polynucleotide having at least about 90%, 95%, or 98% sequence identity to SEQ ID NO: 33. In several embodiments, the engineered NK cells express a chimeric receptor having at least about 90%, 95%, or 98% sequence identity to SEQ ID NO: 34. In several embodiments, the mbIL15 expressed by the NK cells has at least about 90%, 95%, or 98% sequence identity to SEQ ID NO: 36. Alternative embodiments utilize an mbIL15 having at least 95% sequence identity to SEQ ID NO: 38.

In several embodiments, the first and second dose are administered 6-8 days apart and the second and third dose are administered 6-8 days apart. In several embodiments, each dose comprises 1.5×10⁹ NK cells and wherein the second dose is administered about 7 days after the first dose and wherein the third dose is administered about 7 days after the second dose.

In several embodiments, the dosing regimen provided for herein are for treatment of a cancer, such as a blood cancer. In several embodiments, the cancer is Relapsed/Refractory (R/R) Acute Myeloid Leukemia (AML) or Higher Risk Myelodysplastic Syndrome (MDS). In several embodiments, the subject has R/R AML and has received at least 1 but not more than 2 lines of previous standard anti-leukemia therapy. In several embodiments, the subject has fms-like tyrosine kinase 3 (FLT3)-mutated and/or isocitrate dehydrogenase (IDH)1/2-mutated disease and has received at least 1, but not more than 3, lines of prior therapy. In some embodiments, the subject is eligible to be classified as intermediate, high, or very high risk MDS and has relapsed and/or refractory MDS. In several embodiments, the subject has received at least 1 but not more than 2 lines of previous standard anti-MDS therapy. In several embodiments, the subject has less than about 5% blasts in a blood sample. In several embodiments, the subject has a white blood cell count of less than or equal to 25×10⁹ WBC/L. In several embodiments, the subject does not exhibit evidence of leukemic meningitis or known active central nervous system disease and/or does not have peripheral leukocytosis with greater than or equal to 20,000 blasts/μL.

In several embodiments, the dosing regimen provided for herein are for treatment of a cancer, such as a solid tumor. In several embodiments, the cancer is a liver tumor, including intrahepatic cholangiocarcinoma, and liver tumors that are secondary metastases from colorectal cancer.

In several embodiments of the dosing regimens provided for herein, the engineered NK cells are allogeneic with respect to the subject. In several embodiments, the dosing regimen results in one or more of a reduction in blast cell counts, an increase in platelet counts, and an increase in neutrophil counts.

In several embodiments, a dosing regimen for the treatment of Relapsed/Refractory (R/R) Acute Myeloid Leukemia (AML) or Higher Risk Myelodysplastic Syndrome (MDS), is provided and comprises, consists of, or consists essentially of at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, wherein the third dose is administered to the subject between 5-10 days after the second dose, wherein each of the first, second and third doses comprise at least 1.5×10⁹ NK cells, wherein the engineered NK cells are allogeneic with respect to the subject and are engineered to a chimeric receptor that binds ligands of the natural killer cell group 2D (NKG2D) and has at least about 80% sequence identity to SEQ ID NO: 34, and wherein the first dosing cycle is initiated after the subject has received at least three doses of cytosine arabinoside (Ara-C) and at least three doses of fludarabine, and wherein the first dosing cycle is optionally followed by one or more additional dosing cycle.

In several embodiments, there is provided a method for the treatment of Relapsed/Refractory (R/R) Acute Myeloid Leukemia (AML) or Higher Risk Myelodysplastic Syndrome (MDS), comprising administering to a subject having R/R AML or MDS at least three doses of cytosine arabinoside (Ara-C) and at least three doses of fludarabine, wherein the amount of Ara-C administered ranges from about 1 to 5 g/m²/day and the amount of fludarabine administered ranges from about 20 and 40 mg/m²/day, administering to the subject at least a first, a second, and a third dose of genetically engineered NK cells, wherein the first dose is administered to the subject after the last dose of Ara-C and fludarabine, wherein the second dose is administered to the subject between 6-8 days after the first time point, wherein the third dose is administered to the subject between 6-8 days after the second dose, wherein each of the first, second and third doses comprise at least 1.5×10⁹ NK cells, wherein the engineered NK cells are allogeneic with respect to the subject and are engineered to a chimeric receptor that binds ligands of the natural killer cell group 2D (NKG2D) and has at least about 80% sequence identity to SEQ ID NO: 34, and wherein the first dosing cycle is optionally followed by one or more additional dosing cycle.

In several embodiments, the method further comprises evaluating at least one metric related to progression or regression of the status of the R/R AML or MDS to determine whether to administer an additional dosing cycle. In several embodiments, the subject has relapsed and/or refractory acute myeloid leukemia and has received at least 1 but not more than 2 lines of previous standard anti-leukemia therapy. In several embodiments, the subject has fms-like tyrosine kinase 3 (FLT3)-mutated and/or isocitrate dehydrogenase (IDH)1/2-mutated disease and has received at least 1, but not more than 3, lines of prior therapy. In several embodiments, the subject is eligible to be classified as intermediate, high, or very high risk MDS and has relapsed and/or refractory MDS. In several embodiments, the subject has received at least 1 but not more than 2 lines of previous standard anti-MDS therapy. In several embodiments, the subject has less than about 5% blasts in a blood sample, has a white blood cell count of less than or equal to 25×10⁹ WBC/L and/or does not exhibit evidence of leukemic meningitis or known active central nervous system disease and/or does not have peripheral leukocytosis with greater than or equal to 20,000 blasts/μL

In several embodiments, there is provided for herein a dosing regimen for cancer immunotherapy, comprising at least a first dosing cycle, wherein the first dosing cycle is made up of a first dose of genetically engineered natural killer (NK) cells as provided for herein and a second dose of genetically engineered NK cells, wherein the first dose is administered to a subject in need of cancer immunotherapy at a first time point, wherein the second dose is administered to the subject between 5-10 days after the first time point, wherein each of the first and second dose comprise at least 1.5×10⁸ NK cells (or at least 3×10⁶/kg for subject under 50 kg), wherein the first dosing cycle is initiated after the subject has undergone a lymphodepletion process in order to reduce native immune cell numbers, and wherein the first dosing cycle is optionally followed by a second, or greater, dosing cycle.

Additional embodiments provide for a dosing regimen for cancer immunotherapy, comprising at least a first dosing cycle, wherein the first dosing cycle consists of a first dose of genetically engineered natural killer (NK) cells and a second dose of genetically engineered NK cells, wherein the first dose is administered to a subject in need of cancer immunotherapy at a first time point, wherein the second dose is administered to the subject between 5-10 days after the first time point, wherein each of the first and second dose comprise at least 1.5×10⁸ NK cells, wherein the first dosing cycle is initiated after the subject has undergone a lymphodepletion process in order to reduce native immune cell numbers, and wherein the first dosing cycle is optionally followed by a second, or greater, dosing cycle.

According to several embodiments, the dosing cycle is between about 14 days and about 35 days, or longer, and the subject is optionally evaluated with respect to at least one metric of the cancer at end of the dosing cycle or at more distant time points (e.g., on an ongoing basis) to determine if an additional dosing cycle should be initiated. In several embodiments, the dosing cycle is about 21 days. In several embodiments, the dosing cycle is about 28 days.

According to several embodiments, the first and second doses of engineered NK cells are administered to the subject prior to the subject's native immune cell population recovering from the lymphodepletion process. This advantageously allows a greater effector:target cell ratio, based on the subject's native immune cell count not yet having recovered and diluting the population numbers of engineered NK cells. In several embodiments, the first and second doses of engineered NK cells are administered to the subject within about 14 to 21 days after the first time point. In several embodiments, the first and second doses of engineered NK cells are administered to the subject within about 14 days after the first time point.

In several embodiments, there is provided a dosing regimen for cancer immunotherapy, comprising at least a first dosing cycle, wherein the first dosing cycle is made up of a first dose of genetically engineered natural killer (NK) cells and a second dose of genetically engineered NK cells, wherein the first dose of the cycle is administered to a subject in need of cancer immunotherapy at a first time point, wherein the second dose is administered to the subject between 5-10 days after the first time point, wherein each of the first and second dose comprise at least 1.5×10⁸ NK cells, or at least 3×10⁶/kg for subject under 50 kg, wherein the first dosing cycle is initiated after the subject has undergone a lymphodepletion process in order to reduce native immune cell numbers, and wherein the second dose of the first dosing cycle is administered prior to the subject's native immune cell population recovering from the lymphodepletion process.

Also provided for herein is a dosing regimen for cancer immunotherapy, comprising at least a first dosing cycle, wherein the first dosing cycle is made up of a first dose of genetically engineered natural killer (NK) cells and a second dose of genetically engineered NK cells, wherein the first dose of the cycle is administered to a subject in need of cancer immunotherapy at a first time point, wherein the second dose is administered to the subject between 5-10 days after the first time point, wherein, in combination, first and second doses result in administration of between about 300 million and about 3 billion of the engineered NK cells, wherein the first dosing cycle is initiated after the subject has undergone a lymphodepletion process in order to reduce native immune cell numbers, and wherein the second dose of the first dosing cycle is administered within about 14 to 21 days of the first time point. In several embodiments, wherein the first and second dose each comprise about 1.5×10⁸ NK cells, wherein the first and second dose each comprise about 4.5×10⁸ NK cells, or wherein the first and second dose each comprise about 1.5×10⁹ NK cells.

In several embodiments, the lymphodepletion process is a standard chemotherapy lymphodepletion process. In several embodiments, the lymphodepletion process comprises at least two doses of cyclophosphamide and at least two doses of fludarabine. In several embodiments, the lymphodepletion process comprises three doses of cyclophosphamide and three doses of fludarabine, wherein the first of the doses of cyclophosphamide and fludarabine are administered 5 days prior to the initiation of the dosing cycle, wherein the second of the doses of cyclophosphamide and fludarabine are administered 4 days prior to the initiation of the dosing cycle, and wherein the third of the doses of cyclophosphamide and fludarabine are administered 3 days prior to the initiation of the dosing cycle. In several embodiments, about two days are allowed to lapse between the third dose of cyclophosphamide and fludarabine and initiation of the dosing cycle. In several embodiments, the cyclophosphamide is administered in an amount between about 100 and 600 mg/m² and the fludarabine is administered in an amount between about 10 and 60 mg/m². In several embodiments, the cyclophosphamide is administered in an amount between about 200 and 400 mg/m² and the fludarabine is administered in an amount between about 20 and 40 mg/m². In several embodiments, the cyclophosphamide is administered in an amount of about 300 mg/m² and the fludarabine is administered in an amount of about 30 mg/m².

According to embodiments provided for herein, the dosing regimen provides that at least a portion of the engineered NK cells is engineered to express a chimeric receptor comprising a domain that binds ligands of the natural killer cell group 2D (NKG2D), a transmembrane domain, and a cytotoxic signaling complex. In several embodiments, the cytotoxic signaling complex comprises an OX40 subdomain and a CD3zeta subdomain. In several embodiments, the wherein the genetically engineered NK cells also express membrane-bound interleukin 15 (mbIL15).

In several embodiments, the engineered NK cells express a chimeric receptor encoded by a polynucleotide having at least 80%, 85%, 90%, 95%, 96%, 97%, 98%, 99%, or more, sequence identity to SEQ ID NO: 33. In several embodiments, the engineered NK cells express a chimeric receptor having at least 80%, 85%, 90%, 95%, 96%, 97%, 98%, 99%, or more, sequence identity to SEQ ID NO: 34. In several embodiments, the mbIL15 has at least 80%, 85%, 90%, 95%, 96%, 97%, 98%, 99%, or more, sequence identity to SEQ ID NO: 36 and/or 38.

In several embodiments, the first and second dose comprise at least 1.5×10⁸ NK cells and wherein the first and second dose are administered 6-8 days apart. In several embodiments, the first and second dose comprises 1.5×10⁸ NK cells and wherein the first and second dose are administered about 7 days apart. In several embodiments, the first and second dose comprise at least 4.5×10⁸ NK cells and wherein the first and second dose are administered 6-8 days apart. In several embodiments, the first and second dose comprises 4.5×10⁸ NK cells and wherein the first and second dose are administered about 7 days apart. In several embodiments, the first and second dose comprise at least 1.5×10⁹ NK cells and wherein the first and second dose are administered 6-8 days apart. In several embodiments, the first and second dose comprises 1.5×10⁹ NK cells and wherein the first and second dose are administered about 7 days apart.

In several embodiments, the dosing regimen is configured for the treatment of a blood cancer. In several embodiments, the blood cancer is Relapsed/Refractory (R/R) Acute Myeloid Leukemia (AML) or Higher Risk Myelodysplastic Syndrome (MDS).

In several embodiments, the dosing regimen is configured for the treatment of a solid tumor. In several embodiments, the cancer is a liver tumor. In several embodiments, the liver tumor is intrahepatic cholangiocarcinoma. In several embodiments, the liver tumor is one or more secondary metastases from colorectal cancer.

In several embodiments, the engineered NK cells are allogeneic with respect to the subject. In several embodiments, the dosing regimen results in one or more of a reduction in blast cell counts, an increase in platelet counts, and an increase in neutrophil counts.

In several embodiments, there is provided a dosing regimen for cancer immunotherapy, comprising at least a first dosing cycle, wherein the first dosing cycle is made up of at least one dose of genetically engineered natural killer (NK) cells, wherein the first dose is administered to a subject in need of cancer immunotherapy at a first time point, wherein the first dose comprises at least 1×10⁸ NK cells, or 2×10⁶/kg for a subject under 50 kg, wherein at least a portion of the engineered NK cells is engineered to express a chimeric receptor comprising a domain that binds ligands of the natural killer cell group 2D (NKG2D), a transmembrane domain, and a cytotoxic signaling complex, wherein the cytotoxic signaling complex comprises an OX40 subdomain and a CD3zeta subdomain, and wherein the genetically engineered NK cells also express membrane-bound interleukin 15 (mbIL15), wherein the first dosing cycle is initiated after the subject has undergone a lymphodepletion process in order to reduce native immune cell numbers, and wherein the first dosing cycle is optionally followed by a subsequent dosing cycle.

Also provided for herein is a dosing regimen for cancer immunotherapy, comprising: at least a first dosing cycle, wherein the first dosing cycle is made up of at least a first dose of genetically engineered natural killer (NK) cells, a second dose of genetically engineered NK cells, and a second dose of genetically engineered NK cells, wherein the first dose is administered to a subject in need of cancer immunotherapy at a first time point, wherein the second dose is administered to the subject between 5-10 days after the first time point, wherein the third dose is administered to the subject between 5-10 days after the second time point, wherein each of the first, second, and third dose comprise at least 1×10⁸ NK cells, or at least 2×10⁶/kg for subject under 50 kg, wherein at least a portion of the engineered NK cells is engineered to express a chimeric receptor comprising a domain that binds ligands of the natural killer cell group 2D (NKG2D), a transmembrane domain, and a cytotoxic signaling complex, wherein the cytotoxic signaling complex comprises an OX40 subdomain and a CD3zeta subdomain, and wherein the genetically engineered NK cells also express membrane-bound interleukin 15 (mbIL15), wherein the first dosing cycle is initiated after the subject has undergone a lymphodepletion process in order to reduce native immune cell numbers, and wherein the first dosing cycle is optionally followed by a second, or greater, dosing cycle.

In several embodiments, the lymphodepletion process is a standard chemotherapy lymphodepletion process. In several embodiments, the lymphodepletion process comprises at least two doses of cyclophosphamide and at least two doses of fludarabine. In several embodiments, the lymphodepletion process comprises three doses of cyclophosphamide and three doses of fludarabine, wherein the first of the doses of cyclophosphamide and fludarabine are administered 5 days prior to the initiation of the dosing cycle, wherein the second of the doses of cyclophosphamide and fludarabine are administered 4 days prior to the initiation of the dosing cycle, and wherein the third of the doses of cyclophosphamide and fludarabine are administered 3 days prior to the initiation of the dosing cycle. In several embodiments, about two days are allowed to lapse between the third dose of cyclophosphamide and fludarabine and initiation of the dosing cycle. In several embodiments, the cyclophosphamide is administered in an amount between about 100 and 600 mg/m² and the fludarabine is administered in an amount between about 10 and 60 mg/m². In several embodiments, the cyclophosphamide is administered in an amount between about 200 and 400 mg/m² and the fludarabine is administered in an amount between about 20 and 40 mg/m². In several embodiments, the cyclophosphamide is administered in an amount of about 300 mg/m² and the fludarabine is administered in an amount of about 30 mg/m².

In several embodiments, the dosing cycle ranges from about 14 to about 28 days and the subject is optionally evaluated with respect to at least one metric of the cancer at or after the end of the dosing cycle in order to determine if an additional dosing cycle is warranted.

In several embodiments, the engineered NK cells express a chimeric receptor encoded by a polynucleotide having at least 80%, 85%, 90%, 95%, 96%, 97%, 98%, 99%, or more, sequence identity to SEQ ID NO: 33. In several embodiments, the engineered NK cells express a chimeric receptor having at least 80%, 85%, 90%, 95%, 96%, 97%, 98%, 99%, or more, sequence identity to SEQ ID NO: 34. In several embodiments, the mbIL15 has at least 80%, 85%, 90%, 95%, 96%, 97%, 98%, 99%, or more, sequence identity to SEQ ID NO: 36 and/or 38.

In several embodiments, the first, second, and third doses are 1.0×10⁸ NK cells and wherein the are administered 6-8 days apart. In several embodiments, the first, second, and third doses are about 1.0×10⁸ NK cells and are administered about 7 days apart. In several embodiments, the first, second, and third doses are about 3×10⁸ NK cells and are administered 6-8 days apart. In several embodiments, the first, second, and third doses are about 3×10⁸ NK cells and are administered about 7 days apart. In several embodiments, the first, second, and third doses are about 1×10⁹ NK cells and are administered 6-8 days apart. In several embodiments, the first, second, and third doses are about 1×10⁹ NK cells and are administered about 7 days apart.

In several embodiments, the dosing regimen is for the treatment of a blood cancer. In several embodiments, the blood cancer is Relapsed/Refractory (R/R) Acute Myeloid Leukemia (AML) or Higher Risk Myelodysplastic Syndrome (MDS).

In several embodiments, the dosing regimen is for the treatment of a solid tumor. In several embodiments, the cancer is a liver tumor. In several embodiments, the liver tumor is intrahepatic cholangiocarcinoma. In several embodiments, the liver tumor is a secondary metastasis from colorectal cancer.

### BRIEF DESCRIPTION OF THE DRAWINGS

FIG. 1 depicts non-limiting schematics of polynucleotides encoding cytotoxic receptor constructs comprising a binding moiety for ligands of NKG2D and either encoding mbIL15 (Receptor B) or not (Receptor A).

FIG. 2 depicts non-limiting schematics of polynucleotides encoding cytotoxic receptor constructs comprising an NKG2D receptor domain (e.g., fragment) and either encoding mbIL15 (Receptor B) or not (Receptor A).

FIGS. 3A-3B depicts non-limiting schematics of dosing cycles according to embodiments disclosed herein. FIG. 3A shows a 28 day cycle comprising three dosing events. FIG. 3B shows a 28 day cycle comprising two dosing events.

FIGS. 4A-4B show initial patient response data. FIG. 4A show the change in blast counts in subjects before treatment and after a three-dose regimen. FIG. 4B shows a trace of NK cell count in an example subject during and after a three-dose regimen.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a cytotoxicity-inducing receptor complex. As used herein, the term “cytotoxic receptor complexes” shall be given its ordinary meaning and shall also refer to (unless otherwise indicated), Chimeric Antigen Receptors (CAR), chimeric receptors (also called activating chimeric receptors in the case of NKG2D chimeric receptors). In several embodiments, the cells are further engineered to achieve a modification of the reactivity of the cells against non-tumor tissue and/or other therapeutic cells.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. For example, an immune cell, such as an NK cell or a T cell, may be engineered to include a chimeric receptor such as a NKG2D-ligand-directed chimeric receptor, or engineered to include a nucleic acid encoding said chimeric receptor as described herein. Additional embodiments relate to engineering a second set of cells to express another cytotoxic receptor complex, such as an NKG2D chimeric receptor complex as disclosed herein.

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are also provided for herein polynucleotides, polypeptides, and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. Also provided are engineered immune cells (e.g., NK cells and/or T cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., NK cells and/or T cells) expressing such bi-specific constructs (in some embodiments the first and second ligand binding domain target the same ligand). Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric antigen receptor, such as a NKG2D ligand-directed chimeric receptor, or a nucleic acid encoding the chimeric receptor. In several embodiments, the cells are optionally engineered to co-express a membrane-bound interleukin 15 (mbIL15) domain. As discussed in more detail below, in several embodiments, the therapeutic cells, are further genetically modified enhance the cytotoxicity and/or persistence of the cells. In several embodiments, the genetic modification enhances the ability of the cell to resist signals emanating from the tumor microenvironment that would otherwise cause a reduced efficacy or shortened lifespan of the therapeutic cells.

### Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake cellular material, or entire cells, followed by digestion and destruction of the engulfed cellular material. In several embodiments, monocytes are used in connection with one or more additional engineered cells as disclosed herein. Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) domain.

### Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Several embodiments of the methods and compositions disclosed herein relate to lymphocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) domain.

### T Cells for Immunotherapy

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) domain. In several embodiments, immortalized NK cells are used and are subject to engineering, as disclosed herein. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally includes a membrane-bound interleukin 15 (mbIL15) domain.

### Induced Pluripotent Stem Cells

In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the cells are engineered to express a homing moiety and/or a cytotoxic receptor complex. In several embodiments, iPSCs are used in combination with one or more additional engineered cell type disclosed herein. Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### Extracellular Domains (Tumor Binder)

Some embodiments of the compositions and methods described herein relate to a chimeric receptor that includes an extracellular domain that comprises a tumor-binding domain (also referred to as an antigen-binding protein or antigen-binding domain) as described herein. Several embodiments of the compositions and methods described herein relate to a chimeric receptor that includes an extracellular domain that comprises a ligand binding domain that binds a ligand expressed by a tumor cell (also referred to as an activating chimeric receptor) as described herein. The ligand binding domain, depending on the embodiment, targets for example MICA, MICB, ULBP1, ULBP2, ULBP3, ULBP4, ULBP5, and ULBP6 (among others).

In some embodiments, the antigen-binding domain is derived from or comprises wild-type or non-wild-type sequence of an antibody, an antibody fragment, an scFv, a Fv, a Fab, a (Fab′)2, a single domain antibody (SDAB), a vH or vL domain, a camelid VHH domain, or a non-immunoglobulin scaffold such as a DARPIN, an affibody, an affilin, an adnectin, an affitin, a repebody, a fynomer, an alphabody, an avimer, an atrimer, a centyrin, a pronectin, an anticalin, a kunitz domain, an Armadillo repeat protein, an autoantigen, a receptor or a ligand. In some embodiments, the tumor-binding domain contains more than one antigen binding domain.

### Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Non-limiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be produced by the modification of intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions (C) are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain”, refers to the smaller of the two types of polypeptide chains present in antibody molecules in their naturally occurring conformations. Kappa (K) and lambda (A) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (p), delta (A), gamma (γ), alpha (a), and epsilon (s), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type III (Fn3)(see U.S. Pat. No. 6,703,199, which describes fibronectin polypeptide mini bodies). An antibody fragment may include a Fab, Fab′, F(ab′)2, and/or Fv fragment that contains at least one CDR of an immunoglobulin that is sufficient to confer specific antigen binding to a cancer antigen (e.g., CD19). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)2 fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDR-H1, CDR-H2 and CDR-H3) and three light chain variable region CDRs (CDR-L1, CDR-L2 and CDR-L3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FW1, CDR1, FW2, CDR2, FW3, CDR3, FW4. For heavy chain variable regions, the order is typically: FW-H1, CDR-H1, FW-H2, CDR-H2, FW-H3, CDR-H3, and FW-H4 from N-terminus to C-terminus. For light chain variable regions, the order is typically: FW-L1, CDR-L1, FW-L2, CDR-L2, FW-L3, CDR-L3, FW-L4 from N-terminus to C-terminus. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT@(the international ImMunoGeneTics information system; Lefranc et al, Dev. Comp. Immunol. 29:185-203; 2005) and AHo (Honegger and Pluckthun, J. Mol. Biol. 309(3):657-670; 2001). The binding domains disclosed herein may utilize CDRs defined according to any of these systems. For any given embodiment containing more than one CDR, the CDRs may be defined in accordance with any of Kabat, Chothia, extended, IMGT, Paratome, AbM, and/or conformational definitions, or a combination of any of the foregoing. Any of the CDRs, either separately or within the context of variable domains, can be interpreted by one of skill in the art under any of these numbering systems as appropriate. One or more CDRs may be incorporated into a molecule either covalently or noncovalently to make it an antigen-binding protein.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

There is also provided, in some embodiments, antigen-binding proteins with more than one binding site. In several embodiments, the binding sites are identical to one another while in some embodiments the binding sites are different from one another. For example, an antibody typically has two identical binding sites, while a “bispecific” or “bifunctional” antibody has two different binding sites. The two binding sites of a bispecific antigen-binding protein or antibody will bind to two different epitopes, which can reside on the same or different protein targets. In several embodiments, this is particularly advantageous, as a bispecific chimeric antigen receptor can impart to an engineered cell the ability to target multiple tumor markers. For example, MICA, MICB, ULBP1, ULBP2, ULBP3, ULBP4, ULBP5, and ULBP6, among others, and an additional tumor marker, such as CD70, CD123, CD19, Her2, mesothelin, Claudin 6, BCMA, EGFR, or any other marker disclosed herein or appreciated in the art as a tumor specific antigen or tumor associated antigen can be bound by a bispecific antibody.

Natural Killer Group Domains that Bind Tumor Ligands

In several embodiments, engineered immune cells such as NK cells are leveraged for their ability to recognize and destroy tumor cells. NK cells express both inhibitory and activating receptors on the cell surface. Inhibitory receptors bind self-molecules expressed on the surface of healthy cells (thus preventing immune responses against “self” cells), while the activating receptors bind ligands expressed on abnormal cells, such as tumor cells. When the balance between inhibitory and activating receptor activation is in favor of activating receptors, NK cell activation occurs and target (e.g., tumor) cells are lysed.

Natural killer Group 2 member D (NKG2D) is an NK cell activating receptor that recognizes a variety of ligands expressed on cells. The surface expression of various NKG2D ligands is generally low in healthy cells but is upregulated upon, for example, malignant transformation. Non-limiting examples of ligands recognized by NKG2D include, but are not limited to, MICA, MICB, ULBP1, ULBP2, ULBP3, ULBP4, ULBP5, and ULBP6, as well as other molecules expressed on target cells that control the cytolytic or cytotoxic function of NK cells. In several embodiments, T cells are engineered to express an extracellular domain to binds to one or more tumor ligands and activate the T cell. For example, in several embodiments, T cells are engineered to express an NKG2D receptor as the binder/activation moiety. In several embodiments, engineered cells as disclosed herein are engineered to express another member of the NKG2 family, e.g., NKG2A, NKG2C, and/or NKG2E. Combinations of such receptors are engineered in some embodiments. Moreover, in several embodiments, other receptors are expressed, such as the Killer-cell immunoglobulin-like receptors (KIRs).

In several embodiments, cells are engineered to express a cytotoxic receptor complex comprising a full length NKG2D as an extracellular component to recognize ligands on the surface of tumor cells (e.g., liver cells). In one embodiment, full length NKG2D has the nucleic acid sequence of SEQ ID NO: 27. In several embodiments, the full length NKG2D, or functional fragment thereof is human NKG2D. Additional information about chimeric receptors for use in the presently disclosed methods and compositions can be found in PCT Patent Publication No. WO/2018/183385, which is incorporated in its entirety by reference herein.

In several embodiments, cells are engineered to express a cytotoxic receptor complex comprising a functional fragment of NKG2D as an extracellular component to recognize ligands on the surface of tumor cells or other diseased cells. In one embodiment, the functional fragment of NKG2D has the nucleic acid sequence of SEQ ID NO: 25. In several embodiments, the fragment of NKG2D has at least 70%, at least 75%, at least 80%, at least 85%, at least 90%, or at least 95% sequence identity with full-length wild-type NKG2D. In several embodiments, the fragment may have one or more additional mutations from SEQ ID NO: 25, but retains, or in some embodiments, has enhanced, ligand-binding function. In several embodiments, the functional fragment of NKG2D comprises the amino acid sequence of SEQ ID NO: 26. In several embodiments, the NKG2D fragment is provided as a dimer, trimer, or other concatemeric format, such embodiments providing enhanced ligand-binding activity. In several embodiments, the sequence encoding the NKG2D fragment is optionally fully or partially codon optimized. In one embodiment, a sequence encoding a codon optimized NKG2D fragment comprises the sequence of SEQ ID NO: 28. Advantageously, according to several embodiments, the functional fragment lacks its native transmembrane or intracellular domains but retains its ability to bind ligands of NKG2D as well as transduce activation signals upon ligand binding. A further advantage of such fragments is that expression of DAP10 to localize NKG2D to the cell membrane is not required. Thus, in several embodiments, the cytotoxic receptor complex encoded by the polypeptides disclosed herein does not comprise DAP10. In several embodiments, immune cells, such as NK or T cells (e.g., non-alloreactive T cells engineered according to embodiments disclosed herein), are engineered to express one or more chimeric receptors that target, for example CD70, CD19, CD123, Her2, mesothelin, Claudin 6, BCMA, EGFR, and an NKG2D ligand, such as MICA, MICB, ULBP1, ULBP2, ULBP3, ULBP4, ULBP5, and/or ULBP6. Such cells, in several embodiments, also co-express mbIL15.

In several embodiments, the cytotoxic receptor complexes are configured to dimerize. Dimerization may comprise homodimers or heterodimers, depending on the embodiment. In several embodiments, dimerization results in improved ligand recognition by the cytotoxic receptor complexes (and hence the NK cells expressing the receptor), resulting in a reduction in (or lack) of adverse toxic effects. In several embodiments, the cytotoxic receptor complexes employ internal dimers, or repeats of one or more component subunits. For example, in several embodiments, the cytotoxic receptor complexes may optionally comprise a first NKG2D extracellular domain coupled to a second NKG2D extracellular domain, and a transmembrane/signaling region (or a separate transmembrane region along with a separate signaling region).

In several embodiments, the various domains/subdomains are separated by a linker such as, a GS3 linker (SEQ ID NOs: 15 and 16, nucleotide and protein, respectively) is used (or a GSn linker). Other linkers used according to various embodiments disclosed herein include, but are not limited to those encoded by SEQ ID NOs: 17, 19, 21 or 23. In several embodiments, other linkers comprise the peptide sequence of one of SEQ ID NOs: 18, 20, 22, 24. This provides the potential to separate the various component parts of the receptor complex along the polynucleotide, which can enhance expression, stability, and/or functionality of the receptor complex.

### Cytotoxic Signaling Complex

Some embodiments of the compositions and methods described herein relate to a chimeric receptor, such as a chimeric receptor directed against an NKG2D ligand, such as MICA, MICB, ULBP1, ULBP2, ULBP3, ULBP4, ULBP5, and/or ULBP6) that includes a cytotoxic signaling complex. As disclosed herein, according to several embodiments, the provided cytotoxic receptor complexes comprise one or more transmembrane and/or intracellular domains that initiate cytotoxic signaling cascades upon the extracellular domain(s) binding to ligands on the surface of target cells.

In several embodiments, the cytotoxic signaling complex comprises at least one transmembrane domain, at least one co-stimulatory domain, and/or at least one signaling domain. In some embodiments, more than one component part makes up a given domain—e.g., a co-stimulatory domain may comprise two subdomains. Moreover, in some embodiments, a domain may serve multiple functions, for example, a transmembrane domain may also serve to provide signaling function.

### Transmembrane Domains

Some embodiments of the compositions and methods described herein relate to chimeric receptors (e.g., tumor antigen-directed CARs and/or ligand-directed chimeric receptors) that comprise a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

In several embodiments, however, the transmembrane domain comprises at least a portion of CD8, a transmembrane glycoprotein normally expressed on both T cells and NK cells. In several embodiments, the transmembrane domain comprises CD8a. In several embodiments, the transmembrane domain is referred to as a “hinge”. In several embodiments, the “hinge” of CD8a has the nucleic acid sequence of SEQ ID NO: 1. In several embodiments, the CD8a hinge is truncated or modified and has at least 70%, at least 75%, at least 80%, at least 85%, at least 90%, at least 95% sequence identity with the CD8a having the sequence of SEQ ID NO: 1. In several embodiments, the “hinge” of CD8a comprises the amino acid sequence of SEQ ID NO: 2. In several embodiments, the CD8a can be truncated or modified, such that it has at least 70%, at least 75%, at least 80%, at least 85%, at least 90%, at least 95% sequence identity with the sequence of SEQ ID NO: 2.

In several embodiments, the transmembrane domain comprises a CD8a transmembrane region. In several embodiments, the CD8a transmembrane domain has the nucleic acid sequence of SEQ ID NO: 3. In several embodiments, the CD8a hinge is truncated or modified and has at least 70%, at least 75%, at least 80%, at least 85%, at least 90%, at least 95% sequence identity with the CD8a having the sequence of SEQ ID NO: 3. In several embodiments, the CD8a transmembrane domain comprises the amino acid sequence of SEQ ID NO: 4. In several embodiments, the CD8a hinge is truncated or modified and has at least 70%, at least 75%, at least 80%, at least 85%, at least 90%, at least 95% sequence identity with the CD8a having the sequence of SEQ ID NO: 4.

Taken together in several embodiments, the CD8 hinge/transmembrane complex is encoded by the nucleic acid sequence of SEQ ID NO: 13. In several embodiments, the CD8 hinge/transmembrane complex is truncated or modified and has at least 70%, at least 75%, at least 80%, at least 85%, at least 90%, at least 95% sequence identity with the CD8 hinge/transmembrane complex having the sequence of SEQ ID NO: 13. In several embodiments, the CD8 hinge/transmembrane complex comprises the amino acid sequence of SEQ ID NO: 14. In several embodiments, the CD8 hinge/transmembrane complex hinge is truncated or modified and has at least 70%, at least 75%, at least 80%, at least 85%, at least 90%, at least 95% sequence identity with the CD8 hinge/transmembrane complex having the sequence of SEQ ID NO: 14.

In some embodiments, the transmembrane domain comprises a CD28 transmembrane domain or a fragment thereof. In several embodiments, the CD28 transmembrane domain comprises the amino acid sequence of SEQ ID NO: 30. In several embodiments, the CD28 transmembrane domain complex hinge is truncated or modified and has at least 70%, at least 75%, at least 80%, at least 85%, at least 90%, at least 95% sequence identity with the CD28 transmembrane domain having the sequence of SEQ ID NO: 30.

### Co-Stimulatory Domains

Some embodiments of the compositions and methods described herein relate to chimeric receptors (e.g., tumor antigen-directed CARs and/or tumor ligand-directed chimeric receptors) that comprise a co-stimulatory domain. In addition, the various the transmembrane domains and signaling domain (and the combination transmembrane/signaling domains), additional co-activating molecules can be provided, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such co-stimulatory domains are engineered to be membrane bound, acting as autocrine stimulatory molecules (or even as paracrine stimulators to neighboring cells).

In several embodiments, the NK cells disclosed herein are engineered to express interleukin 15 (IL15, IL-15). In some embodiments, the IL15 is expressed from a separate cassette on the construct comprising any one of the CARs disclosed herein. In some embodiments, the IL15 is expressed in the same cassette as any one of the CARs disclosed herein, optionally separated by a cleavage site, for example, a proteolytic cleavage site or a T2A, P2A, E2A, or F2A self-cleaving peptide cleavage site. In some embodiments, the IL15 is a membrane-bound IL15 (mbIL15). In some embodiments, the mbIL15 comprises a native IL15 sequence, such as a human native IL15 sequence, and at least one transmembrane domain. In some embodiments, the native IL15 sequence is encoded by a sequence having at least 85%, at least 90%, at least 95% sequence identity to SEQ ID NO: 11. In some embodiments, the native IL15 sequence comprise a peptide sequence having at least 85%, at least 90%, at least 95% sequence identity to SEQ ID NO: 12. In some embodiments, the at least one transmembrane domain comprises a CD8 transmembrane domain. In some embodiments, the mbIL15 may comprise additional components, such as a leader sequence and/or a hinge sequence. In some embodiments, the leader sequence is a CD8 leader sequence. In some embodiments, the hinge sequence is a CD8 hinge sequence.

In some embodiments, the tumor antigen-directed CARs and/or tumor ligand-directed chimeric receptors are encoded by a polynucleotide that encodes for one or more cytosolic protease cleavage sites. Such sites are recognized and cleaved by a cytosolic protease, which can result in separation (and separate expression) of the various component parts of the receptor encoded by the polynucleotide. In some embodiments, the tumor antigen-directed CARs and/or tumor ligand-directed chimeric receptor are encoded by a polynucleotide that encodes for one or more self-cleaving peptides, for example a T2A cleavage site, a P2A cleavage site, an E2A cleavage site, and/or an F2A cleavage site. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site has the nucleic acid sequence of SEQ ID NO: 9. In several embodiments, T2A cleavage site can be truncated or modified, such that it has at least 70%, at least 75%, at least 80%, at least 85%, at least 90%, at least 95% sequence identity with the sequence of SEQ ID NO: 9. In several embodiments, the T2A cleavage site comprises the amino acid sequence of SEQ ID NO: 10. In several embodiments, the T2A cleavage site is truncated or modified and has at least 70%, at least 75%, at least 80%, at least 85%, at least 90%, at least 95% sequence identity with the T2A cleavage site having the sequence of SEQ ID NO: 10.

In several embodiments, NK cells are engineered to express membrane-bound interleukin 15 (mbIL15). In such embodiments, mbIL15 expression on the NK enhances the cytotoxic effects of the engineered NK cell by enhancing the proliferation and/or longevity of the NK cells. In several embodiments, the mbIL15 is encoded by the same polynucleotide as the CAR. In some embodiments, mbIL15 is encoded by a polynucleotide comprising the sequence of SEQ ID NO: 11 and a sequence that encodes for a transmembrane domain. In some embodiments, mbIL15 comprises the amino acid sequence of SEQ ID NO: 12 functionally coupled to an amino acid sequence of a transmembrane domain. In several embodiments, mbIL15 has the nucleic acid sequence of SEQ ID NO: 1188. In several embodiments, mbIL15 can be truncated or modified, such that it has at least 70%, at least 75%, at least 80%, at least 85%, at least 90%, at least 95% sequence identity with the sequence of SEQ ID NO: 1188. In several embodiments, the mbIL15 comprises the amino acid sequence of SEQ ID NO: 1189. In several embodiments, the mbIL15 is truncated or modified and has at least 70%, at least 75%, at least 80%, at least 85%, at least 90%, at least 95% sequence identity with the mbIL15 having the sequence of SEQ ID NO: 1189. Membrane-bound IL15 sequences are explored in PCT publications WO 2018/183385 and WO 2020/056045, each of which is hereby expressly incorporated by reference in its entirety and pertaining to membrane-bound IL15 sequences.

### Signaling Domains

Some embodiments of the compositions and methods described herein relate to a chimeric receptor (e.g., tumor antigen-directed CARs and/or tumor ligand-directed chimeric receptors) that includes a signaling domain. For example, immune cells engineered according to several embodiments disclosed herein may comprise at least one subunit of the CD3 T cell receptor complex (or a fragment thereof). In several embodiments, the signaling domain comprises the CD3zeta subunit. In several embodiments, the CD3zeta is encoded by the nucleic acid sequence of SEQ ID NO: 7. In several embodiments, the CD3zeta can be truncated or modified, such that it has at least 70%, at least 75%, at least 80%, at least 85%, at least 90%, at least 95% sequence identity with the CD3zeta having the sequence of SEQ ID NO: 7. In several embodiments, the CD3zeta domain comprises the amino acid sequence of SEQ ID NO: 8. In several embodiments, the CD3zeta domain is truncated or modified and has at least 70%, at least 75%, at least 80%, at least 85%, at least 90%, at least 95% sequence identity with the CD3zeta domain having the sequence of SEQ ID NO: 8.

In several embodiments, unexpectedly enhanced signaling is achieved through the use of multiple signaling domains whose activities act synergistically. For example, in several embodiments, the signaling domain further comprises an OX40 domain. In several embodiments, the OX40 domain is an intracellular signaling domain. In several embodiments, the OX40 intracellular signaling domain has the nucleic acid sequence of SEQ ID NO: 5. In several embodiments, the OX40 intracellular signaling domain can be truncated or modified, such that it has at least 70%, at least 75%, at least 80%, at least 85%, at least 90%, at least 95% sequence identity with the OX40 having the sequence of SEQ ID NO: 5. In several embodiments, the OX40 intracellular signaling domain comprises the amino acid sequence of SEQ ID NO: 6. In several embodiments, the OX40 intracellular signaling domain is truncated or modified and has at least 70%, at least 75%, at least 80%, at least 85%, at least 90%, at least 95% sequence identity with the OX40 intracellular signaling domain having the sequence of SEQ ID NO: 6. In several embodiments, OX40 is used as the sole transmembrane/signaling domain in the construct, however, in several embodiments, OX40 can be used with one or more other domains. For example, combinations of OX40 and CD3zeta are used in some embodiments. By way of further example, combinations of CD28, OX40, 4-1 BB, and/or CD3zeta are used in some embodiments.

In several embodiments, the signaling domain comprises a 4-1BB domain. In several embodiments, the 4-1 BB domain is an intracellular signaling domain. In several embodiments, the 4-1 BB domain is encoded by the nucleic acid sequence of SEQ ID NO: 29. In several embodiments, the 4-1 BB domain can be truncated or modified, such that it has at least 70%, at least 75%, at least 80%, at least 85%, at least 90%, at least 95% sequence identity with the 4-1-BB domain having the sequence of SEQ ID NO: 29. In several embodiments, the 4-1BB intracellular signaling domain comprises the amino acid sequence of SEQ ID NO: 30. In several embodiments, the 4-1 BB intracellular signaling domain is truncated or modified and has at least 70%, at least 75%, at least 80%, at least 85%, at least 90%, at least 95% sequence identity with the 4-1 BB intracellular signaling domain having the sequence of SEQ ID NO: 30. In several embodiments, 4-1BB is used as the sole transmembrane/signaling domain in the construct, however, in several embodiments, 4-1BB can be used with one or more other domains. For example, combinations of 4-1BB and CD3zeta are used in some embodiments. By way of further example, combinations of CD28, OX40, 4-1 BB, and/or CD3zeta are used in some embodiments.

In several embodiments, the signaling domain comprises a CD28 domain. In several embodiments the CD28 domain is an intracellular signaling domain. In several embodiments, the CD28 intracellular signaling domain is encoded by the nucleic acid sequence of SEQ ID NO: 31. In several embodiments, the CD28 intracellular signaling domain can be truncated or modified, such that it has at least 70%, at least 75%, at least 80%, at least 85%, at least 90%, at least 95% sequence identity with the CD28 intracellular signaling domain having the sequence of SEQ ID NO: 32. In several embodiments, the CD28 intracellular signaling domain comprises the amino acid sequence of SEQ ID NO: 32. In several embodiments, the CD28 intracellular signaling domain is truncated or modified and has at least 70%, at least 75%, at least 80%, at least 85%, at least 90%, at least 95% sequence identity with the CD28 intracellular signaling domain having the sequence of SEQ ID NO: 32. In several embodiments, CD28 is used as the sole transmembrane/signaling domain in the construct, however, in several embodiments, CD28 can be used with one or more other domains. For example, combinations of CD28 andCD3zeta are used in some embodiments. By way of further example, combinations of CD28, OX40, 4-1 BB, and/or CD3zeta are used in some embodiments.

### Cytotoxic Receptor Complex Constructs

Some embodiments of the compositions and methods described herein relate to chimeric receptors, such as an activating chimeric receptor (ACR) that targets ligands of NKG2D. The expression of these cytotoxic receptor complexes in immune cells, such as genetically modified non-alloreactive T cells and/or NK cells, allows the targeting and destruction of particular target cells, such as cancerous cells. Non-limiting examples of such cytotoxic receptor complexes are discussed in more detail below.

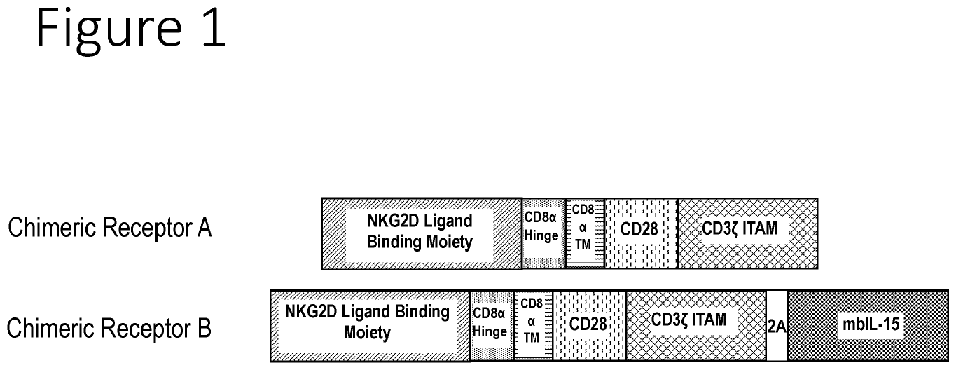

In several embodiments, there is provided a polynucleotide encoding a tumor binder/CD8hinge-CD8TM/OX40/CD3zeta chimeric receptor complex (see FIG. 1, Chimeric Receptor A). The polynucleotide comprises or is composed of a NKG2D ligand binding moiety, a CD8a hinge, a CD8a transmembrane domain, an OX40 domain, a CD3zeta domain. In several embodiments, the polynucleotide further encodes a 2A cleavage site, and an mbIL-15 domain as described herein (see FIG. 1, Chimeric Receptor B, representing the polynucleotide structure where a single polynucleotide encodes both the receptor and the mbIL15). In several embodiments, this receptor complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, the chimeric receptor comprises an amino acid sequence having at least 70%, 75%, 80%, 85%, 90%, 91%, 92%, 93%, 94%, 95%, 96%, 97%, 98%, 99%, or more, or a range defined by any two of the aforementioned percentages, identical to the sequence of one or more of the SEQ IDs provided for herein, or a portion thereof (e.g. a portion excluding the mbIL15 sequence and/or self-cleaving peptide sequence).

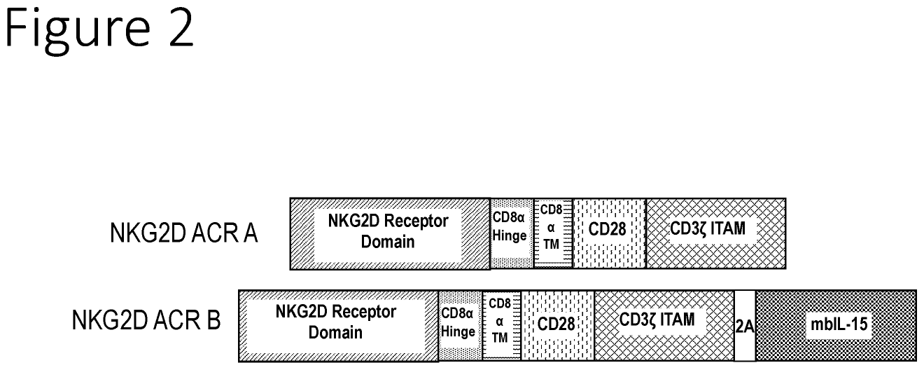

In several embodiments, there is provided a polynucleotide encoding an NKG2D/CD8a hinge/CD8a transmembrane domain/OX40/CD3zeta activating chimeric receptor complex (see FIG. 2, NKG2D ACR A). The polynucleotide comprises or is composed of a fragment of the NKG2D receptor capable of binding a ligand of the NKG2D receptor, a CD8alpha hinge, a CD8a transmembrane domain, an OX40 domain, and a CD3zeta domain as described herein. In several embodiments, this receptor complex is encoded by a nucleic acid molecule comprising the nucleic acid sequence of SEQ ID NO: 33. In yet another embodiment, this chimeric receptor is encoded by the amino acid sequence of SEQ ID NO: 34. In some embodiments, the sequence of the chimeric receptor may vary from SEQ ID NO: 32 or 33, but remains, depending on the embodiment, at least 70%, at least 75%, at least 80%, at least 85%, at least 90%, or at least 95% identical to SEQ ID NO: 33 or 34. In several embodiments, while the chimeric receptor may vary from SEQ ID NO: 33 or 34, the chimeric receptor retains, or in some embodiments, has enhanced, NK cell activating and/or cytotoxic function. Additionally, in several embodiments, this construct can optionally be co-expressed with mbIL15, such as the mbIL15 encoded by SEQ ID NO: 35 or 37 (FIG. 2, NKG2D ACR B, representing the polynucleotide structure where a single polynucleotide encodes both the receptor and the mbIL15). In several embodiments, the mbIL15 is comprises the amino acid sequence of SEQ ID NO: 36 or 38. In some embodiments, the sequence of the mbIL15 may vary from SEQ ID NO: 36 or 38, but remains, depending on the embodiment, at least 70%, at least 75%, at least 80%, at least 85%, at least 90%, or at least 95% identical to SEQ ID NO: 36 or 38

Additional information about chimeric receptors for use in the presently disclosed methods and compositions can be found in PCT Patent Publication No. WO 2018/183385, filed Mar. 27, 2018, which is incorporated in its entirety by reference herein.

### Methods of Treatment

Some embodiments relate to a method of treating, ameliorating, inhibiting, or preventing cancer with a cell or immune cell comprising a chimeric antigen receptor and/or an activating chimeric receptor, as disclosed herein. In some embodiments, the method includes treating or preventing cancer. In some embodiments, the method includes administering a therapeutically effective amount of immune cells expressing a tumor-directed chimeric antigen receptor and/or tumor-directed chimeric receptor as described herein. Examples of types of cancer that may be treated as such are described herein.

Disclosed herein are methods of treating cancer in a subject. In some embodiments, the methods comprise administering to the subject any one of the NKG2D ligand binding domains disclosed herein, any one of the chimeric receptors disclosed herein, or any one of the cells disclosed herein, or any combination thereof.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Advantageously, the non-alloreactive engineered T cells disclosed herein further enhance one or more of the above. Administration can be by a variety of routes, including, without limitation, intravenous, intra-arterial, subcutaneous, intramuscular, intrahepatic, intraperitoneal and/or local delivery to an affected tissue.

Also disclosed herein are uses of any one of the NKG2D ligand binding domains disclosed herein, any one of the chimeric receptors disclosed herein, any one of the cells disclosed herein, or any combination thereof for the treatment of cancer.

Also disclosed herein are uses of any one of the NKG2D ligand binding domains disclosed herein, any one of the chimeric receptors disclosed herein, any one of the cells disclosed herein, or any combination thereof in the manufacture of a medicament for the treatment of cancer.

### Administration and Dosing

Further provided herein are methods of treating a subject having cancer, comprising administering to the subject a composition comprising immune cells (such as NK and/or T cells) engineered to express a cytotoxic receptor complex as disclosed herein. For example, some embodiments of the compositions and methods described herein relate to use of a tumor-directed chimeric antigen receptor and/or tumor-directed chimeric receptor, or use of cells expressing a tumor-directed chimeric antigen receptor and/or tumor-directed chimeric receptor, for treating a cancer patient. Uses of such engineered immune cells for treating cancer are also provided.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; (xi) an enhancement, improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein. Advantageously, the non-alloreactive engineered T cells disclosed herein further enhance one or more of the above.

Administration can be by a variety of routes, including, without limitation, intravenous, intra-arterial, subcutaneous, intramuscular, intrahepatic, intraperitoneal and/or local delivery to an affected tissue. The cells (in particular, NK cells and/or T cells) engineered to express a chimeric receptor complex described herein can be formulated for parenteral administration by injection, e.g., by bolus injection or infusion. In several embodiments,

Doses of immune cells such as NK and/or T cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁶ cells/kg to about 1×10⁸ cells/kg.

In several embodiments, 1×10⁸ NK cells are administered (2×10⁶/kg for subject under 50 kg) three times over a 28-day cycle. In several embodiments, 3×10⁸ NK cells are administered three times over a 28-day cycle. In several embodiments, 1×10⁹ NK cells are administered three times over a 28-day cycle.

In several embodiments, 1.5×10⁸ NK cells are administered (3×10⁶/kg for a subject under 50 kg) two times over a 28-day cycle. In several embodiments, 4.5×10⁸ NK cells are administered two times over a 28-day cycle. In several embodiments, 1.5×10⁹ NK cells are administered two times over a 28-day cycle.

In several embodiments, 1.5×10⁹ NK cells are administered (3×10⁷/kg for a subject under 50 kg) three times over a 28-day cycle. In several embodiments, 3×10⁹ NK cells are administered three times over a 28-day cycle. In several embodiments, 1.5×10¹⁰ NK cells are administered three times over a 28-day cycle. In several embodiments, at least 4.5×10⁹ NK cells are administered over the cycle.

In several embodiments, the administration of engineered NK cells is preceded by one or more preparatory treatments. In several embodiments, the administration of engineered NK cells is preceded by lymphodepletion. In several embodiments, a combination of chemotherapeutic agents is used for lymphodepletion. In several embodiments, a single chemotherapeutic agent is used for lymphodepletion. In several embodiments, wherein a combination of chemotherapeutic agents is used, agents with different mechanisms of actions are optionally used. In several embodiments, different classes of agents are optionally used. In several embodiments, an antimetabolic agent is used. In several embodiments, the antimetabolic agent inhibits and/or prevents cell replication. In several embodiments, the antimetabolic agent is an altered nucleotide that disrupts DNA replication, making it effective in targeting rapidly dividing tumor cells (such as those in AML or myelodysplastic syndrome (MDS)). In several embodiments, cytosine arabinoside (Ara-C) is used. In several embodiments, a dose of between about 0.2-about 10 g/m² Ara-C is administered, including doses of about 0.2 g/m², about 0.5 g/m², about 1.0 g/m², about 1.5 g/m², about 2.0 g/m², about 2.5 g/m², about 3.0 g/m², about 3.5 g/m², about 4.0 g/m², about 5.0 g/m², about 6.0 g/m², about 7.0 g/m², about 8.0 g/m², about 9.0 g/m², about 10.0 about 1.5 g/m², or any dose between those listed. In several embodiments, the dose of Ara-C is given daily for at least about 2 days, about 3 days, about 4 days, about 5 days, about 6 days, or about 7 days. In several embodiments, if necessary, the dose can be split and given, for example, twice daily. In several embodiments, an additional agent is used in combination with the Ara-C. In several embodiments, the additional agent is also an antimetabolite. In several embodiments, the additional agent inhibits one or more of DNA polymerase alpha, ribonucleotide reductase and/or DNA primase, thus inhibiting DNA synthesis. In several embodiments, the additional agent is fludarabine. In several embodiments, a dose of between about 5.0 mg/m²-about 200 mg/m² fludarabine is administered, including doses of about 5.0 mg/m², about 10.0 mg/m², about 15.0 mg/m², about 20.0 mg/m², about 25.0 mg/m², about 30.0 mg/m², about 35.0 mg/m², about 40.0 mg/m², about 45.0 mg/m², about 50.0 mg/m², about 60.0 mg/m², about 70.0 mg/m², about 80.0 mg/m², about 90.0 mg/m², about 100.0 mg/m², about 125.0 mg/m², about 150.0 mg/m², about 175.0 mg/m², about 200.0 mg/m², or any dose between those listed. In several embodiments, the dose of fludarabine is given daily for at least about 2 days, about 3 days, about 4 days, about 5 days, about 6 days, or about 7 days. In several embodiments, if necessary, the dose can be split and given, for example, twice daily. In several embodiments, a combination of fludarabine and Ara-C is used with a daily dose of fludarabine of between about 20 mg/m² and 40 mg/m² and a daily dose of Ara-C of between about 1.5 g/m² and 2.5 g/m². In several embodiments, the combination of fludarabine and Ara-C (or any other agent or agents as disclosed herein) is administered for at least about 5 days, with the administration started about 7 days prior to the first administration of the engineered NK cells (for example day −7 to day −3). In several embodiments, lymphodepletion is started at day −5 prior to administration of engineered NK cells. In several embodiments, this combination advantageously functions not only as a lymphodepletion regimen, but as an anti-cancer agent as well (in addition to the engineered NK cells). In several embodiments, the lymphodepletion regimen works synergistically with the engineered NK cells to provide effect reduction and/or elimination of cancerous cells.

In certain embodiments, a dose of a genetically engineered cell(s) described herein or composition thereof is administered to a subject every day, every other day, every couple of days, every third day, once a week, twice a week, three times a week, or once every two weeks. In other embodiments, two, three or four doses of a genetically engineered cell(s) described herein or composition thereof is administered to a subject every day, every couple of days, every third day, once a week or once every two weeks. In some embodiments, a dose(s) of a genetically engineered cell(s) described herein or composition thereof is administered for 2 days, 3 days, 5 days, 7 days, 14 days, or 21 days. In certain embodiments, a dose of a genetically engineered cell(s) described herein or composition thereof is administered for 1 month, 1.5 months, 2 months, 2.5 months, 3 months, 4 months, 5 months, 6 months or more.

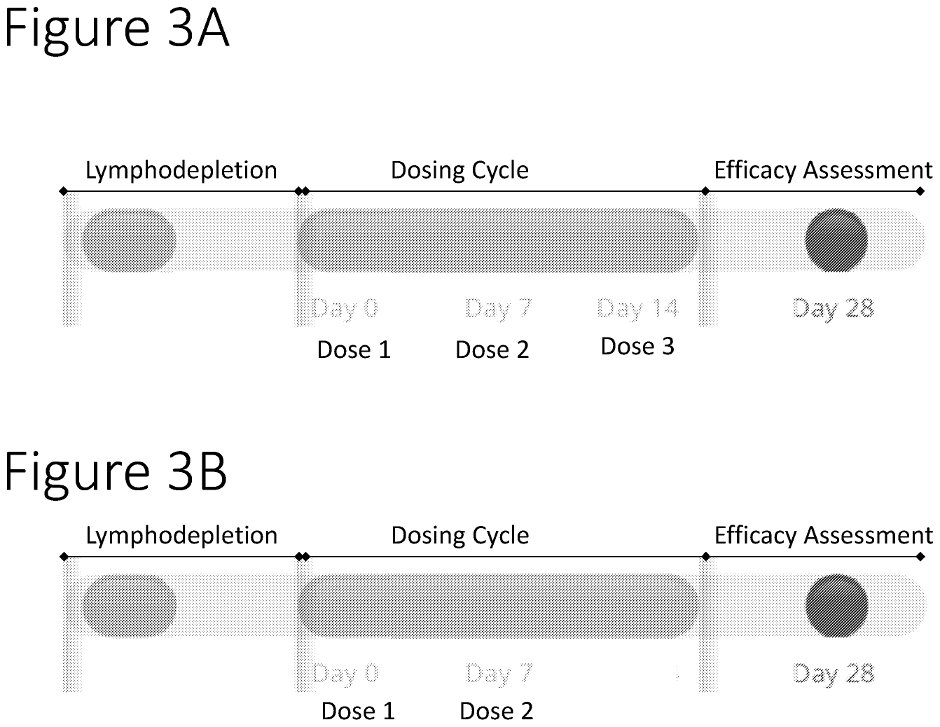

In several embodiments, a subject is subject to lymphodepletion at least one time prior to administration of genetically engineered cells as disclosed herein. In several embodiments, lymphodepletion is performed before one or more additional doses of engineered cells are administered. In several embodiments, a dosing cycle is used that comprises lymphodepletion followed by at least two doses of engineered cells as disclosed herein, with the two doses separated by a time interval. In several embodiments, the time interval is 2, 3, 4, 5, 6, 7, 8, 9, 10, 11, 12, 13, 14, 15, 16, 17, 18, 19, 20, 21, or more days (including intervals falling between the time marking a price interval since the last administration, e.g., 84 hours, or 3.5 days). In several embodiments, the dosing cycle itself is approximately 14, 21, 28, 35, 42 or more days. In several embodiments, three doses are administered, ˜1 week apart from each other. In several embodiments, two doses are administered ˜1 week apart from one another. In several embodiments, a subject receives a first dose on day 0 of the cycle, a second dose on day 7 of the cycle and a third dose on day 14 of the cycle. In several such embodiments, a 28 day cycle is used with primary outcome measures evaluated at day 28 (see e.g., FIG. 3A). In several embodiments, a subject receives a first dose on day 0 of the cycle and a second dose on day 7 of the cycle. In several such embodiments, a 28 day cycle is used with primary outcome measures evaluated at day 28 (See e.g., FIG. 3B). In several embodiments, lymphodepletion is performed prior to the inception of each dosing cycle, if subsequent dosing cycles are required (e.g., the subject requires further treatment). For example, in several embodiments, a subject undergoes lymphodepletion, receives a plurality of doses of engineered cells according to a cycle, is evaluated at the end of the cycle time and, if deemed necessary undergoes a second lymphodepletion followed by a second dosing cycle. In several embodiments, fludarabine/cyclophosphamide is used to achieve lymphodepletion. In several embodiments, cyclophosphamide (300 mg/m²) and fludarabine (30 mg/m²) are administered daily for 3 days. Depending on the embodiment, different concentrations may be used. For example, in several embodiments, a dose of cyclophosphamide of 500 mg/m² is used with the fludarabine. In such embodiments where multiple dosing cycles are used, a first and a second dosing cycle need not be the same (e.g., a first cycle may have 2 doses, while a second uses three doses). Depending on the subject 1, 2, 3, 4, 5, 6, 7, 8, 9, 10, or more dosing cycles are performed.

Depending on the embodiment, various types of cancer can be treated. In several embodiments, the cancer being treated is acute myeloid leukemia (AML). In several embodiments, the cancer being treated is myelodysplastic syndrome. In several embodiments, hepatocellular carcinoma is treated. In several embodiments, intrahepatic cholangiocarcinoma or other liver tumor, for example, secondary metastases from colorectal cancer are treated. Additional embodiments provided for herein include treatment or prevention of the following non-limiting examples of cancers including, but not limited to, acute lymphoblastic leukemia (ALL), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, glioblastoma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS. 1-38 (or combinations of two or more of SEQ ID NOS: 1-38) and that also exhibit one or more of the functions as compared with the respective SEQ ID NOS. 1-38 (or combinations of two or more of SEQ ID NOS: 1-38) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA. In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein.

### Cancer Types

Some embodiments of the compositions and methods described herein relate to administering immune cells comprising a tumor-directed chimeric antigen receptor and/or tumor-directed chimeric receptor to a subject with cancer. Various embodiments provided for herein include treatment or prevention of the following non-limiting examples of cancers. Examples of cancer include, but are not limited to, acute myeloid leukemia (AML), acute lymphoblastic leukemia (ALL), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

### Cancer Targets

Some embodiments of the compositions and methods described herein relate to immune cells comprising a chimeric receptor that targets a cancer antigen, such as MICA, MICB, ULBP1, ULBP2, ULBP3, ULBP4, ULBP5, and/or ULBP6. Additional non-limiting examples of target antigens include: CD70, CD5, CD19; CD123; CD22; CD30; CD171; CS1 (also referred to as CD2 subset 1, CRACC, SLAMF7, CD319, and 19A24); TNF receptor family member B cell maturation (BCMA); CD38; DLL3; G protein coupled receptor class C group 5, member D (GPRC5D); epidermal growth factor receptor (EGFR) CD138; prostate-specific membrane antigen (PSMA); Fms Like Tyrosine Kinase 3 (FLT3); KREMEN2 (Kringle Containing Transmembrane Protein 2), ALPPL2, Claudin 4, Claudin 6, C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(I-4)bDGlcp(I-I)Cer);); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-I)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMI, PTK7, gpNMB, CDH1-CD324, DLL3, CD276/B7H3, ILI IRa, IL13Ra2, CD179b-IGLII, TCRgamma-delta, NKG2D, CD32 (FCGR2A), Tn ag, Timl-/HVCR1, CSF2RA (GM-CSFR-alpha), TGFbetaR2, Lews Ag, TCR-betal chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudinl 8.2 (CLD18A2 or CLDN18A.2)), P-glycoprotein, STEAP1, Livl, Nectin-4, Cripto, gpA33, BST1/CD157, low conductance chloride channel, and the antigen recognized by TNT antibody.

### EXAMPLES

The following are non-limiting descriptions of experimental methods and materials that will be used in examples disclosed below.

### Example 1—First Dosing Regimen for NK Cell Immunotherapy

As discussed in more detail herein, certain cancer types express selected markers in an elevated manner. In several embodiments, cytotoxic receptor constructs are generated according to sequences disclosed herein in order to specifically target a given cancer. For example, many cancers express elevated levels of ligands for the NKG2D receptor. Thus, as discussed in detail above, in several embodiments, NKG2D-ligand-directed cytotoxic receptor constructs are provided. In several embodiments, the polynucleotides encoding those constructs are engineered to bi-cistronically express mbIL15. In several embodiments, a dosing regimen will be tested to evaluate the efficacy of cells expressing such constructs. In several embodiments, the cells engineered to express the construct are NK cells. In several embodiments, the NK cells are off the shelf allogeneic engineered NK cells (derived from an unrelated donor) and will compared against matched doses of haplo-matched related donor-derived engineered NK cells. In several embodiments, the engineered NK cells express the cytotoxic receptor encoded by SEQ ID NO: 33 (including degeneracies or codon-optimized versions of SEQ ID NO: 33). In several embodiments, the engineered NK cells express the cytotoxic receptor comprising the amino acid sequence of SEQ ID NO: 34 and mbIL15 comprising the amino acid sequence of SEQ ID NO: 36 or 38.

A dosing regimen will be designed to evaluate three doses of engineered NK cells that are administered three times in a 28-day dosing cycle. The dosing cycle will be preceded by a conditioning phase during which a subject undergoes lymphodepletion (using cyclophosphamide (300 mg/m²) and fludarabine (30 mg/m²) and day −5, day −4, and day −3) At day 0 the subject will receive the first of one of three different doses: 1×10⁸ NK cells (2×10⁶/kg for subject under 50 kg), 3×10⁸ NK cells, or 1×10⁹ NK cells. Dose 2 is administered at day 7 and dose 3 is administered at day 14. At day 28, outcome measures are assessed.

Primary Outcome Measures include: (1) incidence, nature, and severity of treatment related adverse events will be evaluated. An adverse event is any unfavorable and unintended sign including clinically significant abnormal laboratory findings, symptom or disease. This is to be measured 30 days after last dose of the NK cells and (2) proportion of subjects experiencing dose-limiting toxicities (DLTs) of the NK cells, with DLTs defined as adverse events attributable to treatment that occur during Cycle 1 and meet protocol specified criteria. This is to be measured 28 days from first dose of NK cells.

Secondary outcome measures include: (1) assessment of NK cell half-life, measured as the time required for 50% reduction from maximum amount of circulating engineered NK cells. This is to be measured 28 days from first dose of NK cells; (2) NK cell duration of persistence by measuring amount of engineered NK cells in peripheral blood every 3 months after dosing to determine persistence. This will be measured for up to 2 years after last dose of NK cells; (3) evaluation of host immune response against engineered NK cells through serum samples that will be measured for antibodies against the engineered NK cells. This will be measured for up to 2 years after last dose of NK cells; (4) objective response rate to engineered NK cells by measuring the percentage of subjects with complete and partial response. AML subjects will be assessed for anti-tumor activity of engineered NK cells based on the updated ELN criteria (Dohner 2017). Subjects with MDS will be assessed for anti-tumor activity of engineered NK cells based on the IWG criteria with MDS (Cheson 2006). This will be measured 28 days after first dose of engineered NK cells followed up to 2 years after last dose.

Studies will begin with the lowest dose and if the dose clears the regimen with less than 33% toxicities, the next higher dose will be studied.

It is believed that the administration of three doses of engineered NK cells expressing a chimeric receptor construct targeting NKG2D ligands and also expressing mbIL15 will be favorably tolerated and show limited adverse events. It is also believed that the administration of three doses of engineered NK cells expressing a chimeric receptor construct targeting NKG2D ligands and also expressing mbIL15 will result in limited DLTs. It is believed that the NK cells will show an extended half-life as well as enhanced duration of persistence. It is believed that the NK cells will induce limited host immune response and a clinically meaningful objective response rate (e.g.,) reductions in tumor burden.

### Example 2—Second Dosing Regimen for NK Cell Immunotherapy

As discussed in more detail herein, certain cancer types express selected markers in an elevated manner. In several embodiments, cytotoxic receptor constructs are generated according to sequences disclosed herein in order to specifically target a given cancer. For example, many cancers express elevated levels of ligands for the NKG2D receptor. Thus, as discussed in detail above, in several embodiments, NKG2D-ligand-directed cytotoxic receptor constructs are provided. In several embodiments, the polynucleotides encoding those constructs are engineered to bi-cistronically express mbIL15. In several embodiments, a dosing regimen will be tested to evaluate the efficacy of cells expressing such constructs. In several embodiments, the cells engineered to express the construct are NK cells. In several embodiments, the NK cells are off the shelf allogeneic engineered NK cells (derived from an unrelated donor) and will compared against matched doses of haplo-matched related donor-derived engineered NK cells. In several embodiments, the engineered NK cells express the cytotoxic receptor encoded by SEQ ID NO: 33 (including degeneracies or codon-optimized versions of SEQ ID NO: 33). In several embodiments, the engineered NK cells express the cytotoxic receptor comprising the amino acid sequence of SEQ ID NO: 34 and mbIL15 comprising the amino acid sequence of SEQ ID NO: 36 or 38.

A dosing regimen will be designed to evaluate three different doses of engineered NK cells that are administered two times in a 28-day dosing cycle. The dosing cycle will be preceded by a conditioning phase during which a subject undergoes lymphodepletion (using cyclophosphamide (300 mg/m²) and fludarabine (30 mg/m²) and day −5, day −4, and day −3) At day 0 the subject will receive the first of one of three different doses: 1.5×10⁸ NK cells (3×10⁶/kg for subject under 50 kg), 4.5×10⁸ NK cells, or 1.5×10⁹ NK cells. Dose 2 is administered at day 7. At day 28, outcome measures are assessed.

Primary Outcome Measures include: (1) incidence, nature, and severity of treatment related adverse events will be evaluated. An adverse event is any unfavorable and unintended sign including clinically significant abnormal laboratory findings, symptom or disease. This is to be measured 30 days after last dose of the NK cells and (2) proportion of subjects experiencing dose-limiting toxicities (DLTs) of the NK cells, with DLTs defined as adverse events attributable to treatment that occur during Cycle 1 and meet protocol specified criteria. This is to be measured 28 days from first dose of NK cells.

Secondary outcome measures include: (1) assessment of NK cell half-life, measured as the time required for 50% reduction from maximum amount of circulating engineered NK cells. This is to be measured 28 days from first dose of NK cells; (2) NK cell duration of persistence by measuring amount of engineered NK cells in peripheral blood every 3 months after dosing to determine persistence. This will be measured for up to 2 years after last dose of NK cells; (3) evaluation of host immune response against engineered NK cells through serum samples that will be measured for antibodies against the engineered NK cells. This will be measured for up to 2 years after last dose of NK cells; (4) objective response rate to engineered NK cells by measuring the percentage of subjects with complete and partial response. AML subjects will be assessed for anti-tumor activity of engineered NK cells based on the updated ELN criteria (Dohner 2017). Subjects with MDS will be assessed for anti-tumor activity of engineered NK cells based on the IWG criteria with MDS (Cheson 2006). This will be measured 28 days after first dose of engineered NK cells followed up to 2 years after last dose.

Studies will begin with the lowest dose and if the dose clears the regimen with less than 33% toxicities, the next higher dose will be studied.

It is believed that the administration of three doses of engineered NK cells expressing a chimeric receptor construct targeting NKG2D ligands and also expressing mbIL15 will be favorably tolerated and show limited adverse events. It is also believed that the administration of three doses of engineered NK cells expressing a chimeric receptor construct targeting NKG2D ligands and also expressing mbIL15 will result in limited DLTs. It is believed that the NK cells will show an extended half-life as well as enhanced duration of persistence. It is believed that the NK cells will induce limited host immune response and a clinically meaningful objective response rate (e.g.,) reductions in tumor burden.

It is also believed that the two-dose cycle will yield enhanced outcomes as compared to a three-dose cycle. Without being bound by theory, the larger initial load of engineered NK cells in a two-dose cycle allows for a greater effector:target cell ratio. Studies have demonstrated that host NK cell populations begin to recover about 14 to 21 days after Flu/Cy lymphodepletion. This can result in concomitant clearing of infused allogeneic cells. Thus, a two-dose cycle will enable delivery of a maximized number of engineered cells to be administered in the post-lymphodepletion window, resulting in enhanced therapeutic outcomes.

### Example 3—Third Dosing Regimen for NK Cell Immunotherapy

As discussed in more detail herein, certain cancer types express selected markers in an elevated manner, such as ligands for the NKG2D receptor. Thus, as discussed in detail above, in several embodiments, NKG2D-ligand-directed cytotoxic receptor constructs are provided. In several embodiments, a dosing regimen will be tested to evaluate the efficacy of cells expressing such constructs. In several embodiments, the cells engineered to express the construct are NK cells. In several embodiments, the NK cells are off the shelf allogeneic engineered NK cells (derived from an unrelated donor) and will optionally be compared against matched doses of haplo-matched related donor-derived engineered NK cells. In several embodiments, the engineered NK cells express the cytotoxic receptor encoded by a polynucleotide having at least 80% sequence identity to SEQ ID NO: 33 (including degeneracies or codon-optimized versions of SEQ ID NO: 33). In several embodiments, the engineered NK cells express a cytotoxic receptor comprising an amino acid sequence having at least 80% sequence identity to SEQ ID NO: 34. In several embodiments, the immune cells (e.g., NK cells) are also engineered to express mbIL15 comprising an amino acid sequence having at least 80% sequence identity to SEQ ID NO: 36 or 38.

In several embodiments, the patient to receive the NK cell immunotherapy regimen has relapsed and/or refractory acute myeloid leukemia (per standard European LeukemiaNet (ELN) criteria. In several embodiments, the patient will have received at least 1, but at most 3, and preferably at most 2 lines of previous standard anti-leukemia therapy. In several embodiments, subjects in complete remission with minimal residual disease (e.g., less than about 5% blasts) may receive the NK cell immunotherapy regimen. In several embodiments, the patient may have fms-like tyrosine kinase 3 (FLT3)-mutated and/or isocitrate dehydrogenase (IDH)1/2-mutated disease, and will have received at least 1 prior respective targeted therapy but may have received at most up to 4, and preferably at most 3 lines of prior therapy. In several embodiments, the patient will have a white blood cell count of less than or equal to 25×10⁹ WBC/L. In several embodiments, the patients will not receive the NK cell immunotherapy regimen if they have exhibited evidence of leukemic meningitis or known active central nervous system disease and/or have peripheral leukocytosis with greater than or equal to 20,000 blasts/μL (or other evidence of rapidly progressive disease that would preclude the patient from completing at least 1 cycle of treatment)

In several embodiments, the patient to receive the NK cell immunotherapy regimen are classified as intermediate, high, or very high risk MDS per World Health Organization classification and Revised International Prognostic Scoring System and have relapsed and/or refractory MDS. In several embodiments, the patient will have received at least 1, but at most 3, and preferably at most 2 lines of previous standard anti-MDS therapy.

A dosing regimen will be designed comprising three doses of engineered NK cells that are administered within in a 28-day dosing cycle. The dosing cycle will be preceded by a conditioning phase during which a subject undergoes lymphodepletion (using cytosine arabinoside (Ara-C) (2.0 g/m²/day) and fludarabine (30 mg/m²/day) at day −7, day −6, day −5, day −4, and day −3. At day 0 the subject will receive the first of three doses of NK cells engineered to express a chimeric receptor targeting ligands of NKG2D and also mbIL15. Each dose will be 1.5×10⁹ NK cells. Dose 2 is administered at day 7 and dose 3 is administered at day 14. At day 28, outcome measures are assessed.

Primary Outcome Measures include: (1) incidence, nature, and severity of treatment related adverse events will be evaluated. An adverse event is any unfavorable and unintended sign including clinically significant abnormal laboratory findings, symptom or disease. This is to be measured 30 days after last dose of the NK cells and (2) proportion of subjects experiencing dose-limiting toxicities (DLTs) of the NK cells, with DLTs defined as adverse events attributable to treatment that occur during Cycle 1 and meet protocol specified criteria. This is to be measured 28 days from first dose of NK cells.

Secondary outcome measures include: (1) assessment of NK cell half-life, measured as the time required for 50% reduction from maximum amount of circulating engineered NK cells. This is to be measured 28 days from first dose of NK cells; (2) NK cell duration of persistence by measuring amount of engineered NK cells in peripheral blood every 3 months after dosing to determine persistence. This will be measured for up to 2 years after last dose of NK cells; (3) evaluation of host immune response against engineered NK cells through serum samples that will be measured for antibodies against the engineered NK cells. This will be measured for up to 2 years after last dose of NK cells; (4) objective response rate to engineered NK cells by measuring the percentage of subjects with complete and partial response. AML subjects will be assessed for anti-tumor activity of engineered NK cells based on the updated ELN criteria (Dohner 2017). Subjects with MDS will be assessed for anti-tumor activity of engineered NK cells based on the IWG criteria with MDS (Cheson 2006). This will be measured 28 days after first dose of engineered NK cells followed up to 2 years after last dose.

It is believed that the administration of the three doses of engineered NK cells expressing a chimeric receptor construct targeting NKG2D ligands and also expressing mbIL15 will be favorably tolerated and show limited adverse events. It is also believed that the administration of three doses of engineered NK cells expressing a chimeric receptor construct targeting NKG2D ligands and also expressing mbIL15 will result in limited DLTs. It is believed that the NK cells will show an extended half-life as well as enhanced duration of persistence. It is believed that the NK cells will induce limited host immune response and a clinically meaningful objective response rate (e.g.,) reductions in tumor burden. It is believed that the combination of lymphodepletion using Ara-C and fludarabine, by virtue of the combined effects of lymphodepletion and anti-tumor effects, will further enhance the anti-cancer effect of the engineered NK cells.

### Example 4—Preliminary Outcomes from Three Dose Treatment Regimen

Subjects were treated according to either Example 1 or Example 3 as discussed above. Additional subjects received three doses of either 100 million or 300 million engineered NK cells at each of Day 0, Day 7, and Day 14 (though this dosing cycle is disclosed herein other than in Example 1 or 3). Alternative dosing/timing may be used depending on the embodiment. The median age of the subjects was 60 years of age, with 17 of the subjects diagnosed with AML and 4 with MDS. The median duration of time since diagnosis was 13 months. The median baseline blast percentage was 27% and 15 of the subjects had a neutrophil count (ANC) of less than 1×10⁹ cells/L. Many of the subjects treated according to either Example 1 or Example 3 had been pre-treated with one or more different therapies and/or had multiple relapses of either AML or MDS. Of the treated subjects, the median of the number of prior therapies received was three prior therapies. Of the subjects having AML, each of them had been previously treated with a BCL-2 inhibitor compound. Four of the patients (whether AML of MDS) had received a prior allogeneic cell transplant.

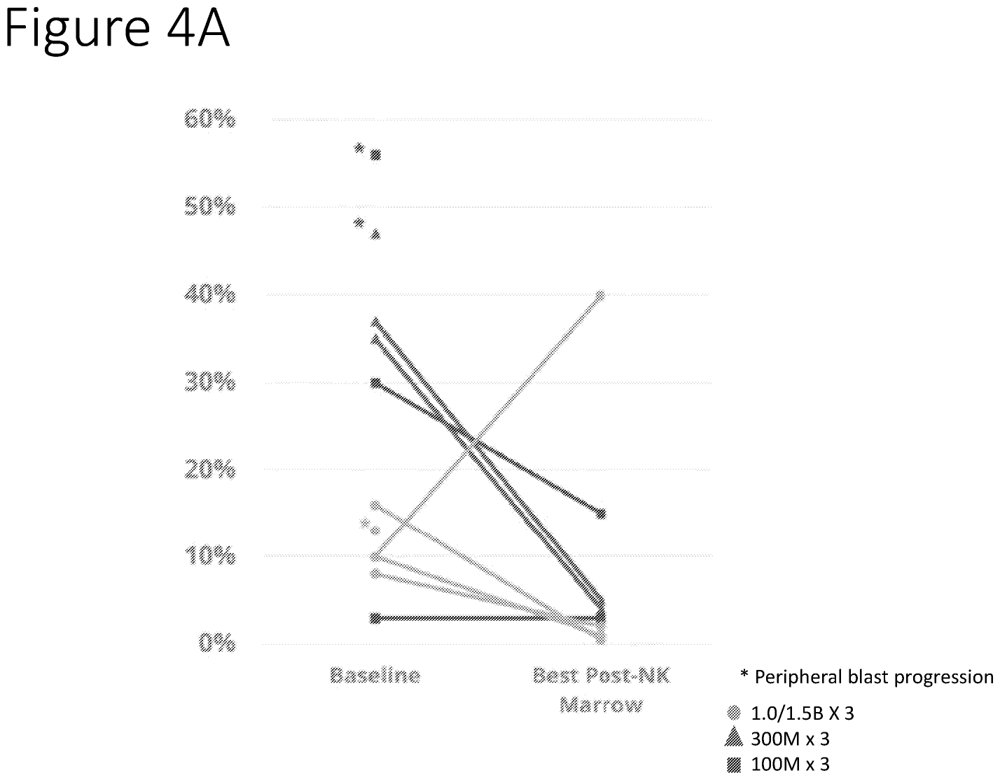

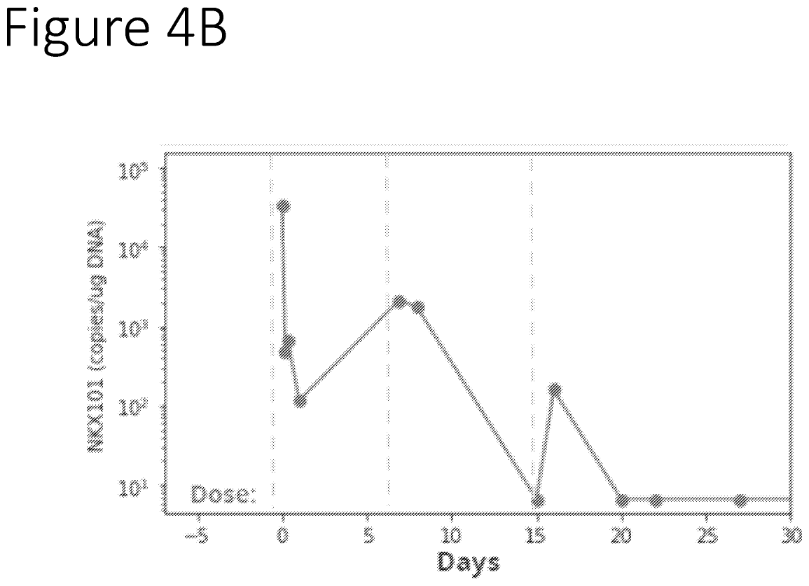

As discussed subjects were subject to lymphodepletion (using cyclophosphamide (300 mg/m²) and fludarabine (30 mg/m²) and day −5, day −4, and day −3). The subjects received three doses of engineered NK cells expressing a chimeric receptor construct targeting NKG2D ligands and also expressing mbIL15. Outcomes were measured at Day 28. Across the subjects dosed, three doses of engineered NK cells expressing a chimeric receptor construct targeting NKG2D ligands and also expressing mbIL15 was well tolerated at each dose level tested. No dose-limiting toxicities were observed. Myelosuppression and infection consistent with lymphodepletion and underlying disease were detected, and represented the most common higher grade reactions. Advantageously, no CAR T-like toxicities were observed at any dose. No subjects experienced cytokine release syndrome, Immune Effector Cell-Associated Neurotoxicity Syndrome (ICANS) or other neurotoxicity, or graft versus host disease. The overall response rate for subjects receiving any of the four doses of NK cells was over 60%. Those receiving either 100 million or 300 million cells at each of the three doses showed nearly 70% overall response rate (either complete response; complete response with incomplete hematologic recovery; morphological leukemia-free state; or partial response). In those patients receiving three doses of either 1×10⁹ or 1.5×10⁹ cells at each dosing point, 60% showed complete response with two of three complete response subjects were observed to be minimal residual disease negative (e.g., no disease was detected after treatment). FIG. 4A shows the change in blast counts from baseline (most recent blast count from the subject prior to first dose). As can be seen, regardless of dose, there is a notable trend for reduction in blast counts. As noted above, three of the 1.0/1.5×10⁹ cell-receiving subjects exhibited complete response, with one of those observed to observed to be minimal residual disease positive and two of those observed to be minimal residual disease negative. One subject (a 68 year old male) who was diagnosed with AML (IDH1 mutation) who was refractory to four prior lines of therapy with a baseline of 8% blasts (by morphology) and 25% (by FISH). After his dosing cycle (which by way of non-limiting example was 1.0×10⁹ engineered NK cells at Day 0, 7 and 14). FIG. 4B shows a graph of the detection of engineered NK cells (by measurement of engineered NK cell DNA) over time for this subject. Engineered NK cells were detected in the blood after each of the three doses (dotted lines). The administered cells exhibited an expected NK-cell like pharmacokinetic profile and clearance by day 20. After the first cycle, the subject was observed to have a complete response and be observed to be minimal residual disease negative. An observation of his bone marrow showed a normocellular phenotype. The engineered NK cells were well tolerated with anemia, neutropenia and decreased platelet count as minor hematologic effects. At a 6-month follow-up, the subject had undergone a consolidative hematopoietic cell transplant and the subject remained with complete response. These initial data demonstrate that engineered NK cells expressing an NKG2D-targeting chimeric receptor and expressing mbIL15 are safe and effective cancer immunotherapy agents, in particular when a patient is dosed on a three-dose regimen as provided for in embodiments herein.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, codon optimization, or other types of modifications.

In accordance with some embodiments described herein, any of the sequences may be used, or a truncated or mutated form of any of the sequences disclosed herein (and/or included in the accompanying sequence listing) may be used and in any combination.

A Sequence Listing in electronic format may be submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences.

1. A method for the treatment of a cancer, comprising administering:
at least a first dosing cycle, wherein the first dosing cycle comprises a first dose of genetically engineered natural killer (NK) cells, a second dose of genetically engineered NK cells, and a third dose of genetically engineered NK cells,
wherein the first dose is administered to a subject having the cancer at a first time point,
wherein the second dose of genetically engineered NK cells is administered to the subject between 5-10 days after the first time point,
wherein the third dose of genetically engineered NK cells is administered to the subject between 5-10 days after the second dose of genetically engineered NK cells;
wherein each of the first, second and third doses of genetically engineered NK cells comprises between 1.0×10⁹ genetically engineered NK cells and 3.0×10⁹ genetically engineered NK cells,
wherein at least a portion of the genetically engineered NK cells is engineered to express a chimeric receptor that binds ligands of the natural killer cell group 2D (NKG2D), and
wherein the first dosing cycle is initiated after the subject has undergone a lymphodepletion process to reduce native immune cell numbers.

2. The method of claim 1, wherein the first, second, and third dose of genetically engineered NK cells each comprise about 1.5×10⁹ NK cells.

3. The method of claim 1, wherein the dosing cycle is between about 14 days and about 35 days.

4-6. (canceled)

7. The method of claim 1, wherein the lymphodepletion process comprises administration of cyclophosphamide and fludarabine.

8. The method of claim 1, wherein:
(i) the lymphodepletion process comprises administration of three doses of cyclosphosphamide and three doses of fludarabine; and
ii (a) about two days are allowed to lapse between the third doses of cyclophosphamide and fludarabine and initiation of the dosing cycle; or
(b) the first doses of cyclophosphamide and fludarabine are administered 5 days prior to the initiation of the dosing cycle, the second doses of cyclophosphamide and fludarabine are administered 4 days prior to the initiation of the dosing cycle, and the third doses of cyclophosphamide and fludarabine are administered 3 days prior to the initiation of the dosing cycle.

9. (canceled)

10. The method of claim 7, wherein the cyclophosphamide is administered in an amount between about 200 and 400 mg/m² and the fludarabine is administered in an amount between about 20 and 40 mg/m².

11. (canceled)

12. The method of claim 1, wherein the lymphodepletion process comprises administration of cytosine arabinoside (Ara-C) and fludarabine.

13. The method of claim 1, wherein:
i) the lymphodepletion process comprises 5 daily doses of Ara-C and 5 daily doses of fludarabine; and
(ii) (a) the first doses of Ara-C and fludarabine are administered 7 days prior to the initiation of the dosing cycle; or
(b) about two days are allowed to lapse between the final doses of Ara-C and fludarabine and initiation of the dosing cycle.

14-15. (canceled)

16. The method of claim 2, wherein the Ara-C is administered in an amount between about 1 and 5 g/m²/day and the fludarabine is administered in an amount between about 20 and 40 mg/m²/day.

17-18. (canceled)

19. The method of claim 1, wherein the cancer is a blood cancer.

20. The method of claim 1, wherein the cancer is Relapsed/Refractory (R/R) Acute Myeloid Leukemia (AML) or Myelodysplastic Syndrome (MDS).

21-27. (canceled)

28. The method of claim 1, wherein the cancer is a solid tumor.

29. (canceled)

30. The method of claim 1, wherein the second and third doses of genetically engineered NK cells are administered to the subject within about 21 days or within about 14 days of the first time point.

31-32. (canceled)

33. The method of claim 1, wherein the chimeric receptor comprises an intracellular signaling domain comprising an OX40 subdomain and a CD3zeta subdomain.

34. The method of claim 1, wherein the genetically engineered NK cells are also engineered to express membrane-bound interleukin 15 (mbIL15).

35. (canceled)

36. The method of claim 1, wherein the genetically engineered NK cells are allogeneic with respect to the subject.

37. The method of claim 1, wherein the first dosing cycle is followed by an additional dosing cycle.

38. A method for the treatment of a cancer, comprising:
(a) administering to a subject having a cancer a lymphodepletion regimen comprising at least two doses of fludarabine,
wherein the dose of fludarabine is from about 20 to about 40 mg/m²/day,
(b) administering to the subject at least a first dose, a second dose, and a third dose of genetically engineered NK cells,
wherein the first dose of genetically engineered NK cells is administered to the subject after the last dose of fludarabine,
wherein the second dose of genetically engineered NK cells is administered to the subject between 6-8 days after the first dose of genetically engineered NK cells,
wherein the third dose of genetically engineered NK cells is administered to the subject between 6-8 days after the second dose of genetically engineered NK cells,
wherein each of the first, second and third doses of genetically engineered NK cells comprises between 1.0×10⁹ genetically engineered NK cells and 3.0×10⁹ genetically engineered NK cells.
wherein the genetically engineered NK cells are allogeneic with respect to the subject and are engineered to express a chimeric receptor that binds ligands of the natural killer cell group 2D (NKG2D).

39. The method of claim 38, wherein each of the first, second and third doses of genetically engineered NK cells comprises about 1.5×10⁹ genetically engineered NK cells.

40. The method of claim 38, wherein the lymphodepletion regimen further comprises at least two doses of cyclophosphamide or at least two doses of cytosine arabinoside (Ara-C).

41-52. (canceled)

53. The method of claim 38, wherein the cancer comprises Relapsed/Refractory (R/R) Acute Myeloid Leukemia (AML) or Higher-Risk-Myelodysplastic Syndrome (MDS).

54-91. (canceled)

92. A method for the treatment of Acute Myeloid Leukemia (AML) comprising administering to a subject having AML a first dosing cycle comprising a first dose, a second dose, and a third dose of genetically engineered natural killer (NK) cells expressing a chimeric receptor that binds ligands of the natural killer cell group 2D (NKG2D), wherein:
the subject was previously administered a lymphodepletion regimen comprising at least two doses of fludarabine and cytosine arabinoside (Ara-C),
the first dose of genetically engineered NK cells is administered to the subject after the last doses of fludarabine and Ara-C,
the second dose of genetically engineered NK cells is administered to the subject between 6-8 days after the first dose of genetically engineered NK cells,
the third dose of genetically engineered NK cells is administered to the subject between 6-8 days after the second dose of genetically engineered NK cells,
each of the first, second and third doses of genetically engineered NK cells comprises between 1.0×10⁹ genetically engineered NK cells and 3.0×10⁹ genetically engineered NK cells,
the genetically engineered NK cells are allogeneic to the subject.

93. The method of claim 92, wherein the AML is Relapsed/Refractory AML (R/R AML).

94. The method of claim 92, wherein each of the first, second, and third doses of genetically engineered NK cells comprises about 1.5×10⁹ genetically engineered NK cells.

95. The method of claim 92, wherein the method comprises administering an additional dosing cycle.
